(19) United States
(12) Patent Application Publication
(10) Pub. No.: US 2011/0105567 A1
(43) Pub. Date: May 5, 2011

(54) Pyrrole Compounds Having Sphingosine-1-Phosphate Receptor Agonist Or Antagonist Biological Activity

(75) Inventors: Richard L. Beard, Newport Beach, CA (US); Hiaqing Yuan, Irvine, CA (US); John E. Donello, Dana Point, CA (US); Ken Chow, Newport Coast, CA (US); Liming Wang, Irvine, CA (US)

(73) Assignee: Allergan, Inc., Irvine, CA (US)

(21) Appl. No.: 12/674,122

(22) PCT Filed: Aug. 21, 2008
(86) PCT No.: PCT/US08/73795

§ 371 Date: Feb. 18, 2010

Related U.S. Application Data
(60) Provisional application No. 60/957,274, filed on Aug. 22, 2007.

(51) Int. Cl. A61K 31/40; C07D 207/34; C07D 401/06; C07D 401/12; C07D 405/06; C07D 207/333; C07D 409/14; C07D 405/12; C07D 413/06; A61K 31/4439; A61K 31/4025; A61K 31/422; A61P 27/06; A61P 27/04; A61P 35/00; A61P 29/02; A61P 17/02; A61P 9/00

(52) U.S. Cl. 514/343; 548/537; 546/279.1; 548/517; 548/540; 548/238; 514/423; 514/422; 514/374

(57) ABSTRACT

Disclosed herein are compounds represented by: therapeutic methods, compositions, and medicaments related thereto are also disclosed.

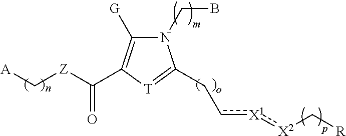

### CROSS-REFERENCE

This application claims the benefit of U.S. Provisional Application Ser. No. 60/957,274, filed Aug. 22, 2007, which is hereby incorporated by reference in its entirety.

### BACKGROUND

Sphingosine is a compound having the chemical structure shown in the general formula described below, in which Y¹ is hydrogen. It is known that various sphingolipids, having sphingosine as a constituent, are widely distributed in the living body including on the surface of cell membranes of cells in the nervous system.

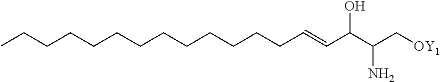

A sphingolipid is one of the lipids having important roles in the living body. A disease called lipidosis is caused by accumulation of a specified sphingolipid in the body. Sphingolipids present on cell membranes function to regulate cell growth; participate in the development and differentiation of cells; function in nerves; are involved in the infection and malignancy of cells; etc. Many of the physiological roles of sphingolipids remain to be solved. Recently the possibility that ceramide, a derivative of sphingosine, has an important role in the mechanism of cell signal transduction has been indicated, and studies about its effect on apoptosis and cell cycle have been reported.

Sphingosine-1-phosphate is an important cellular metabolite, derived from ceramide that is synthesized de novo or as part of the sphingomeyeline cycle (in animals cells). It has also been found in insects, yeasts and plants.

The enzyme, ceramidase, acts upon ceramides to release sphingosine, which is phosphorylated by sphingosine kinase, a ubiquitous enzyme in the cytosol and endoplasmic reticulum, to form sphingosine-1-phosphate. The reverse reaction can occur also by the action of sphingosine phosphatases, and the enzymes act in concert to control the cellular concentrations of the metabolite, which concentrations are always low. In plasma, such concentration can reach 0.2 to 0.9 μM, and the metabolite is found in association with the lipoproteins, especially the HDL. It should also be noted that sphingosine-1-phosphate formation is an essential step in the catabolism of sphingoid bases.

Like its precursors, sphingosine-1-phosphate is a potent messenger molecule that perhaps uniquely operates both intra- and inter-cellularly, but with very different functions from ceramides and sphingosine. The balance between these various sphingolipid metabolites may be important for health. For example, within the cell, sphingosine-1-phosphate promotes cellular division (mitosis) as opposed to cell death (apoptosis), which it inhibits. Intracellularly, it also functions to regulate calcium mobilization and cell growth in response to a variety of extracellular stimuli. Current opinion appears to suggest that the balance between sphingosine-1-phosphate and ceramide and/or sphingosine levels in cells is critical for their viability. In common with the lysophospholipids, especially lysophosphatidic acid, with which it has some structural similarities, sphingosine-1-phosphate exerts many of its extra-cellular effects through interaction with five specific G protein-coupled receptors on cell surfaces. These are important for the growth of new blood vessels, vascular maturation, cardiac development and immunity, and for directed cell movement.

Sphingosine-1 phosphate is stored in relatively high concentrations in human platelets, which lack the enzymes responsible for its catabolism, and it is released into the blood stream upon activation of physiological stimuli, such as growth factors, cytokines, and receptor agonists and antigens. It may also have a critical role in platelet aggregation and thrombosis and could aggravate cardiovascular disease. On the other hand the relatively high concentration of the metabolite in high-density lipoproteins (HDL) may have beneficial implications for atherogenesis. For example, there are recent suggestions that sphingosine-1-phosphate, together with other lysolipids such as sphingosylphosphorylcholine and lysosulfatide, are responsible for the beneficial clinical effects of HDL by stimulating the production of the potent antiatherogenic signaling molecule nitric oxide by the vascular endothelium. In addition, like lysophosphatidic acid, it is a marker for certain types of cancer, and there is evidence that its role in cell division or proliferation may have an influence on the development of cancers. These are currently topics that are attracting great interest amongst medical researchers, and the potential for therapeutic intervention in sphingosine-1-phosphate metabolism is under active investigation.

Fungi and plants have sphingolipids and the major sphingosine contained in these organisms has the formula described below. It is known that these lipids have important roles in the cell growth of fungi and plants, but details of the roles remain to be solved.

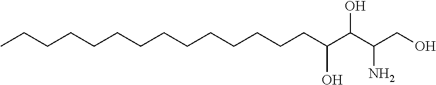

Recently it has been known that derivatives of sphingolipids and their related compounds exhibit a variety of biological activities through inhibition or stimulation of the metabolism pathways. These compounds include inhibitors of protein kinase C, inducers of apoptosis, immuno-suppressive compounds, antifungal compounds, and the like. Substances having these biological activities are expected to be useful compounds for various diseases.

### DESCRIPTION OF THE INVENTION

Disclosed herein is a compound represented by:

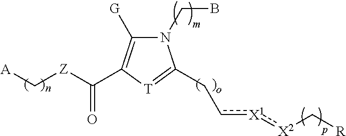

wherein a dashed line represents the presence or absence of a bond;
A and B are independently stable substituted or unsubstituted aryl, or substituted or unsubstituted heteroaryl, wherein A and B independently have a formula C₁₋₁₂H₀₋₂₉N₀₋₄S₀₋₄F₀₋₆Cl₀₋₂Br₀₋₂I₀₋₂;
m, n, o, and p are independently 0, 1, 2, or 3;
R is H; C₁₋₈ non-linear alkyl; C₁₋₈ acyl; C₁₋₈ alkoxycarbonyl; or a stable substituted or unsubstituted heterocycle or phenyl having a formula C₁₋₁₂H₀₋₂₉N₀₋₄O₀₋₃S₀₋₃F₀₋₆Cl₀₋₂I₀₋₂;
Z is CH₂, O, N, or S;
T is CH or N or an alkyl having from 1 to 4 carbon atoms;
G is H, or is a moiety having from 1 to 6 carbon atoms selected from: alkyl wherein one of the carbons may be substituted with S, fluoroalkyl, acyl, hydroxyalkyl, amino or substituted or unsubstituted heteroaryl; and
X¹ and X² are independently a bond,

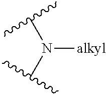

having from 1 to 4 carbon atoms,

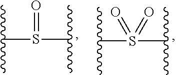

### C═O, —CH═, ═CH—, NH, ═N—, —N═, S, or O;

provided that both X¹ and X² are not bonds.

These compounds are useful for the treatment of diseases or conditions such as glaucoma, dry eye, angiogenesis, cardiovascular conditions and diseases, wounds, and pain. The compound is incorporated into a dosage form or a medicament and administered to the mammal, such as a person, in need thereof. Different types of suitable dosage forms and medicaments are well known in the art, and can be readily adapted for delivery of the compounds disclosed herein.

For the purposes of this disclosure, “treat,” “treating,” or “treatment” refer to the use of a compound, composition, therapeutically active agent, or drug in the diagnosis, cure, mitigation, treatment, or prevention of disease or other undesirable condition.

Unless otherwise indicated, reference to a compound should be construed broadly to include pharmaceutically acceptable salts, prodrugs, tautomers, alternate solid forms, non-covalent complexes, and combinations thereof, of a chemical entity of the depicted structure or chemical name.

A pharmaceutically acceptable salt is any salt of the parent compound that is suitable for administration to an animal or human. A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counter-ions. Salts can form from or incorporate one or more deprotonated acidic groups (e.g. carboxylic acids), one or more protonated basic groups (e.g. amines), or both (e.g. zwitterions).

A prodrug is a compound which is converted to a therapeutically active compound after administration. For example, conversion may occur by hydrolysis of an ester group or some other biologically labile group. Prodrug preparation is well known in the art. For example, “Prodrugs and Drug Delivery Systems,” which is a chapter in Richard B. Silverman, Organic Chemistry of Drug Design and Drug Action, 2d Ed., Elsevier Academic Press: Amsterdam, 2004, pp. 496-557, provides further detail on the subject.

Tautomers are isomers that are in rapid equilibrium with one another. For example, tautomers may be related by transfer of a proton, hydrogen atom, or hydride ion.

Unless stereochemistry is explicitly depicted, a structure is intended to include every possible stereoisomer, both pure or in any possible mixture.

Alternate solid forms are different solid forms than those that may result from practicing the procedures described herein. For example, alternate solid forms may be polymorphs, different kinds of amorphous solid forms, glasses, and the like.

Non-covalent complexes are complexes that may form between the compound and one or more additional chemical species that do not involve a covalent bonding interaction between the compound and the additional chemical species. They may or may not have a specific ratio between the compound and the additional chemical species. Examples might include solvates, hydrates, charge transfer complexes, and the like.

Aryl is an aromatic ring or ring system such as phenyl, naphthyl, biphenyl, and the like.

Heteroaryl is aryl having one or more N, O, or S atoms in the ring, i.e. one or more ring carbons are substituted by N, O, and/or S.

Substituted aryl or heteroaryl is aryl or heteroaryl having one or more substituents attached to the ring instead of hydrogen.

Examples of substituents may include the following subject to the constraints defined herein for that particular moiety having substitutents:
A. Hydrocarbyl, meaning a moiety consisting of carbon and hydrogen only, including, but not limited to:

1. alkyl, such as:
linear alkyl, e.g. methyl, ethyl, n-propyl, n-butyl, n-pentyl, n-hexyl, etc.,
branched alkyl, e.g. iso-propyl, t-butyl and other branched butyl isomers, branched pentyl isomers, etc.,
cycloalkyl, e.g. cyclopropyl, cyclobutyl, cyclopentyl, cyclohexyl, etc.,
combinations of linear, branched, and/or cycloalkyl;

2. alkenyl, e.g. hydrocarbyl having 1 or more double bonds, including linear, branched, or cycloalkenyl

3. alkynyl, e.g. hydrocarbyl having 1 or more triple bonds, including linear, branched, or cycloalkynyl;

4. combinations of alkyl, alkenyl, and/or akynyl
B. alkyl-CN, such as —CH₂—CN, —(CH₂)₂—CN; —(CH₂)₃—CN, and the like;
C. Hydroxy, —OH
D. hydroxyalkyl, i.e. alkyl-OH, such as hydroxymethyl, hydroxyethyl, and the like;
E. ether substituents, including -O-alkyl, alkyl-O-alkyl, and the like;
F. thioether substituents, including —S-alkyl, alkyl-S-alkyl, and the like;
G. amine substituents, including —NH₂, —NH-alkyl, —N-alkyl¹alkyl² (i.e., alkyl¹ and alkyl² are the same or different, and both are attached to N), alkyl-NH₂, alkyl-NH-alkyl, alkyl-N-alkyl¹alkyl², and the like;
H. aminoalkyl, meaning alkyl-amine, such as aminomethyl (—CH₂-amine), aminoethyl, and the like;
I. ester substituents, including —CO₂-alkyl, —CO₂-phenyl, etc.;
J. other carbonyl substituents, including carboxylic acids; aldehydes; ketones, such as acyl, including, acetyl, propionyl, and benzoyl substituents are contemplated;
K. fluorocarbons or hydroflourocarbons such as —CF₃, —CH₂CF₃, etc.; and
L. other nitrogen containing substituents such as —CN and —NO₂,
M. other sulfur containing subsitutents such as thiol, sulfide, sulfonyl or sulfoxide;
N. combinations of the above are also possible, subject to the constraints defined;
O. Alternatively, a substituent may be —F, —Cl, —Br, or —I.

Stable means that the moiety is sufficiently stable to be stored in a bottle at room temperature under a normal atmosphere for at least 12 hours, or stable enough to be useful for any purpose disclosed herein.

If a substituent is a salt, for example of a carboxylic acid or an amine, the counter-ion of said salt, i.e. the ion that is not covalently bonded to the remainder of the molecule is not counted for the purposes of the number of heavy atoms in a substituent. Thus, for example, the salt —CO₂⁻Na⁺ is a stable substituent consisting of 1 carbon atom and 2 oxygen atoms, i.e. sodium is not counted. In another example, the salt —NH(Me)₃⁺Cl⁻ is a stable substituent consisting of 1 nitrogen atom, three carbon atoms, and 10 hydrogen atoms, i.e. chlorine is not counted.

Alkyl is a moiety consisting of carbon and hydrogen having no double bonds, such as linear alkyl, branched alkyl, or cyclic alkyl.

Non-linear alkyl is alkyl that is not linear. Linear alkyl is alkyl having all carbon atoms present as either —CH₂— or —CH₃ and no rings are formed by the carbon atoms. Non-linear alkyl includes at least one carbon atom that is bonded to three or four other carbon atoms, or contains a ring formed by carbon atoms. Examples of non-linear alkyl include iso-propyl, (-butyl, cyclobutyl, cyclopentyl, cyclohexyl, and the like. C₁₋₈ non-linear alkyl is non-linear alkyl having from 1 to 8 carbon atoms.

Acyl is

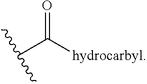

C₁₋₈ acyl is acyl having from 1 to 8 carbon atoms.

Alkoxycarbonyl is

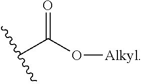

C₁₋₈ alkoxycarbonyl is alkoxycarbonyl having from 1 to 8 carbon atoms.

Aminocarbonyl (i.e., Amide) is

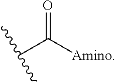

C₁₋₈ aminocarbonyl is aminocarbonyl having from 1 to 8 carbon atoms.

Amino is —NH₂, —NH(hydrocarbyl), or —N(hydrocarbyl)₂, where the two hydrocarbyl moieties may be the same or different, or may form a ring.

Fluoroalkyl is alkyl wherein from 1 to all of the hydrogens that are normally present on alkyl are substituted with fluorine.

A and B are independent, meaning that they may be the same or different from one another.

The formula C₁₋₁₂H₀₋₂₉N₀₋₄O₀₋₄S₀₋₄F₀₋₆Cl₀₋₂Br₀₋₂I₀₋₂ means that the moiety of that formula is composed of the following atoms:
from 1 to 12 carbon atoms;
from 0 to 29 hydrogen atoms;
from 0 to 4 nitrogen atoms;
from 0 to 4 oxygen atoms;
from 0 to 4 sulfur atoms;
from 0 to 6 fluorine atoms;
from 0 to 2 chlorine atoms;
from 0 to 2 bromine atoms; and
from 0 to 2 iodine atoms.

Similarly, the formula C₀₋₁₂H₀₋₂₁N₀₋₄O₀₋₃F₀₋₆Cl₀₋₂Br₀₋₂I₀₋₂ means that the moiety of that formula is composed of the following atoms:
from 1 to 12 carbon atoms;
from 0 to 21 hydrogen atoms;
from 0 to 4 nitrogen atoms;
from 0 to 3 oxygen atoms;
from 0 to 3 sulfur atoms;
from 0 to 6 fluorine atoms;
from 0 to 2 chlorine atoms;
from 0 to 2 bromine atoms; and
from 0 to 2 iodine atoms.

For example, A may be phenyl, or substituted phenyl, such as in one of the structures depicted below.

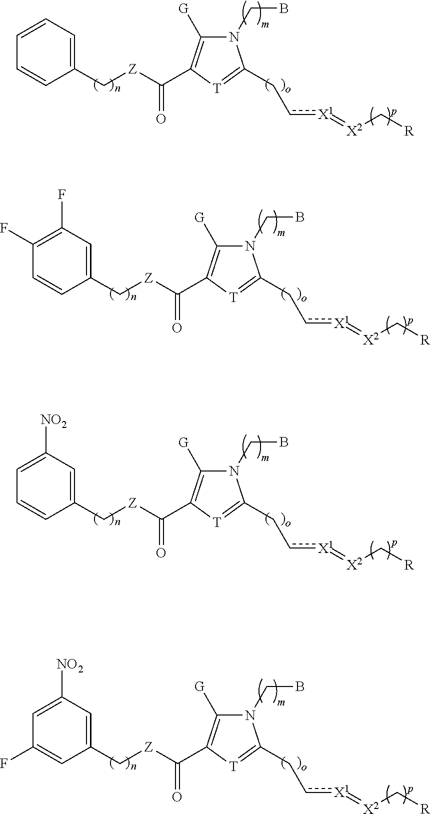

A may also be unsubstituted or substituted pyridinyl, such as in one of the structures depicted below.

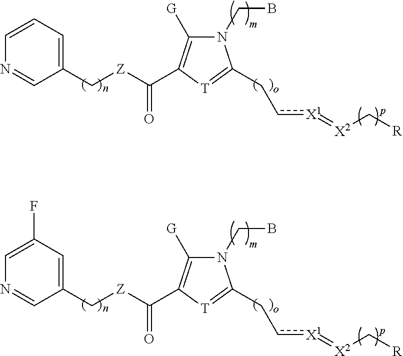

The pyridinyl may be attached in other positions, such as ortho or para to the nitrogen atom, and the pyridinyl may also be substituted.

Other examples of A include substituted and unsubstituted thienyl, furyl, pyrrolyl, imidazolyl, oxazolyl, thiazolyl, triazole, oxadiazole, thiadaizole, and the like.

B may be phenyl, such as in the structure depicted below.

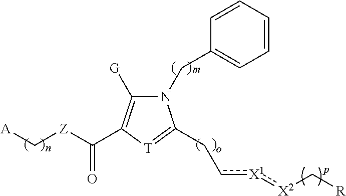

The phenyl may also be substituted.

B may also be pyridinyl, such as in the structure depicted below.

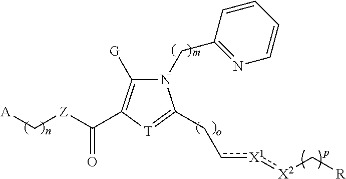

The pyridinyl may be attached in other positions, such as meta or para to the nitrogen atom, and the pyridinyl may also be substituted.

Other examples of B include substituted and unsubstituted thienyl, furyl, pyrrolyl, imidazolyl, oxazolyl, thiazolyl, triazole, oxadiazole, thiadiazole and the like.

In another embodiment, B is phenyl or pyridinyl.

In these compounds, m, n, o, and p are independently 0, 1, 2, or 3. In other words, m, n, o, and p may have the same or different values with respect to one another.

Examples of structures arising from the possible values of m, n, o, and p are depicted below.

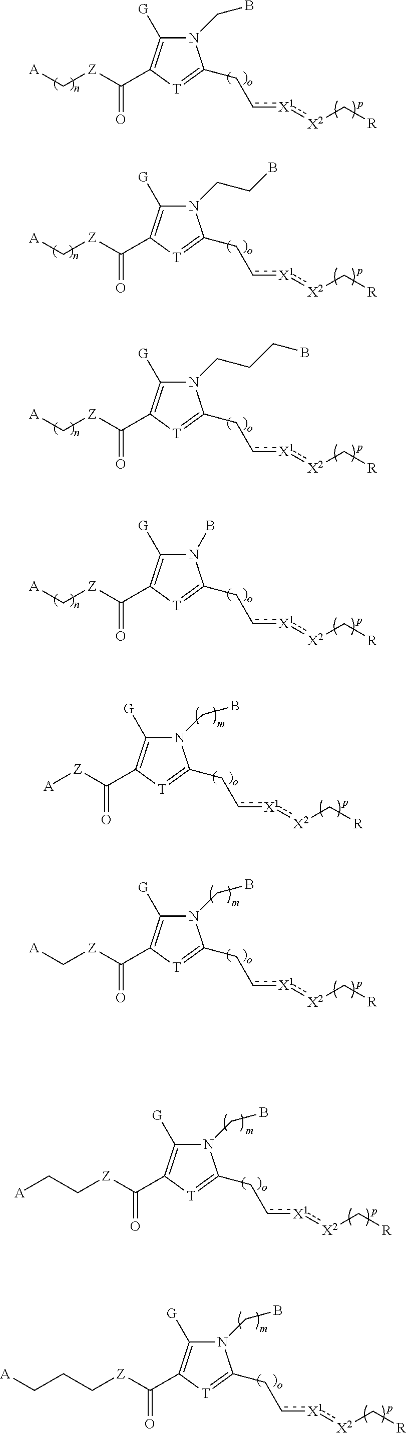

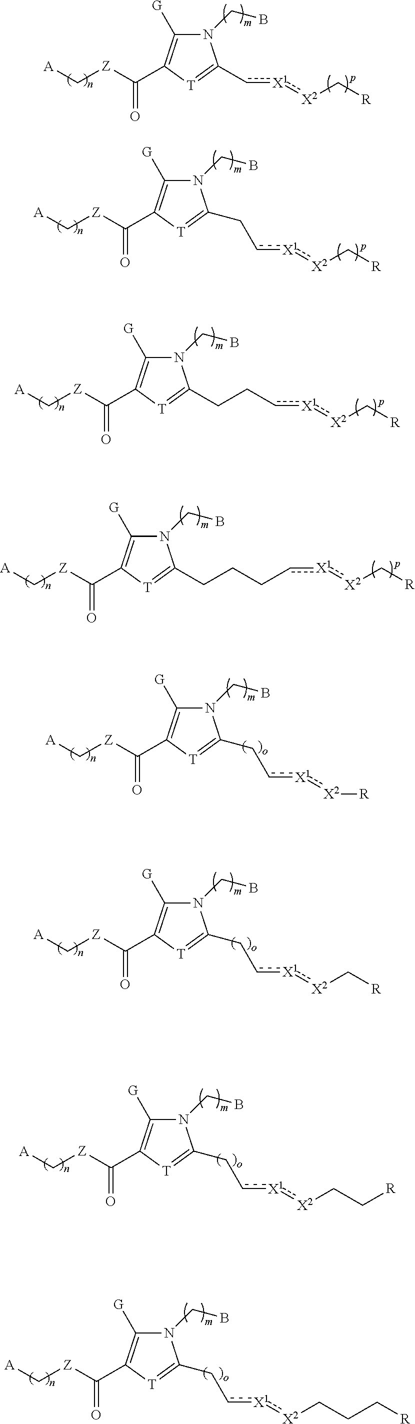

In one embodiment, R is:
methyl, ethyl, iso-propyl, propyl, iso-butyl, cyclobutyl, cyclopentyl, cyclohexyl, or phenyl;

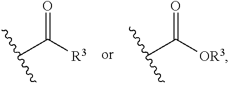

wherein R³ is methyl, ethyl, iso-propyl, propyl, iso-butyl, cyclobutyl, cyclopentyl, cyclohexyl, or phenyl; or heterocycle, including

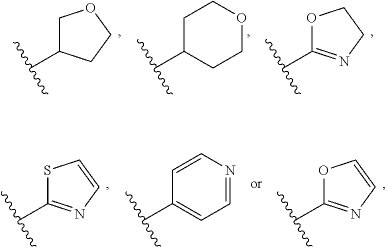

wherein any hydrogen atom may be replaced by a substituent.

In another embodiment, R is substituted phenyl.

Some compounds contemplated according to the present invention are:

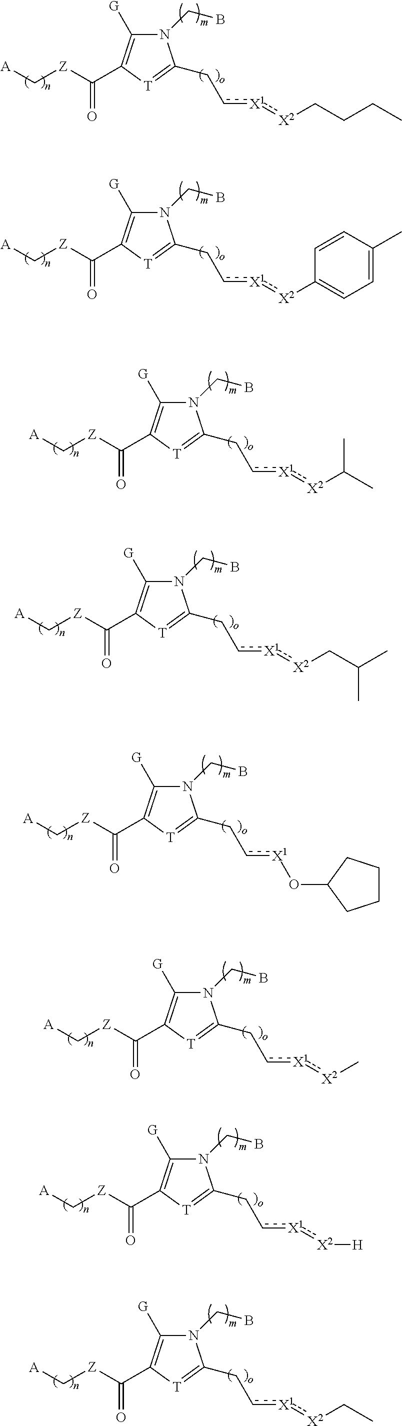

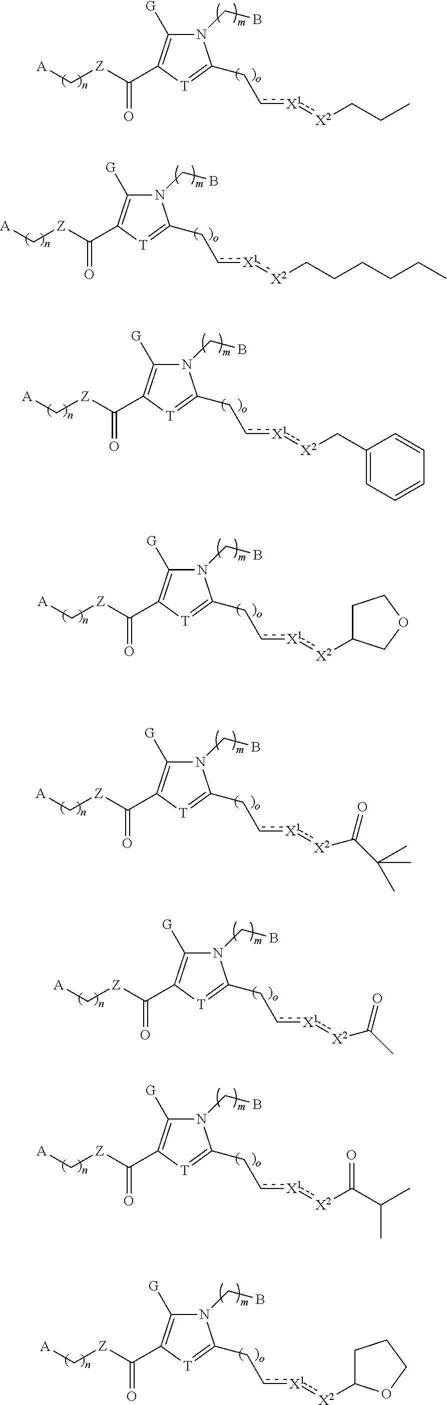

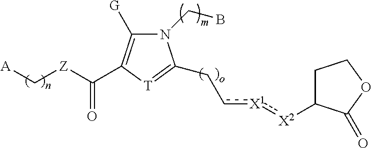

G is H, or is a moiety having from 1 to 6 carbon atoms selected from: alkyl, fluoroalkyl, acyl, hydroxyalkyl, or amino. The —N indicates that if G is an amine it attaches at the nitrogen. Thus, compounds contemplated according to the present invention include:

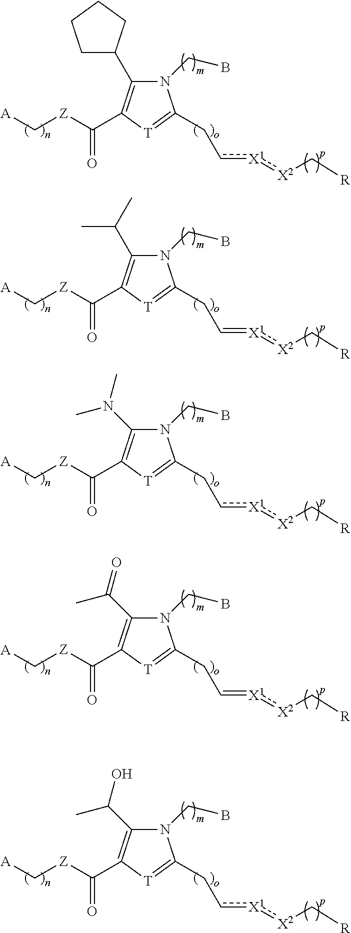

Other examples of G include methyl, ethyl, isobutyl, sec-butyl, tert-butyl, cyclohexyl, cyclic —NC₄H₈, and cyclic —NC₅H₁₀.

X¹ is a bond,

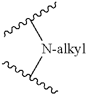

having from 1 to 4 carbon atoms,

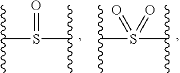

C═O, NH, ═N—, —N═, S, or O. Thus, compounds having the structures below are also contemplated.

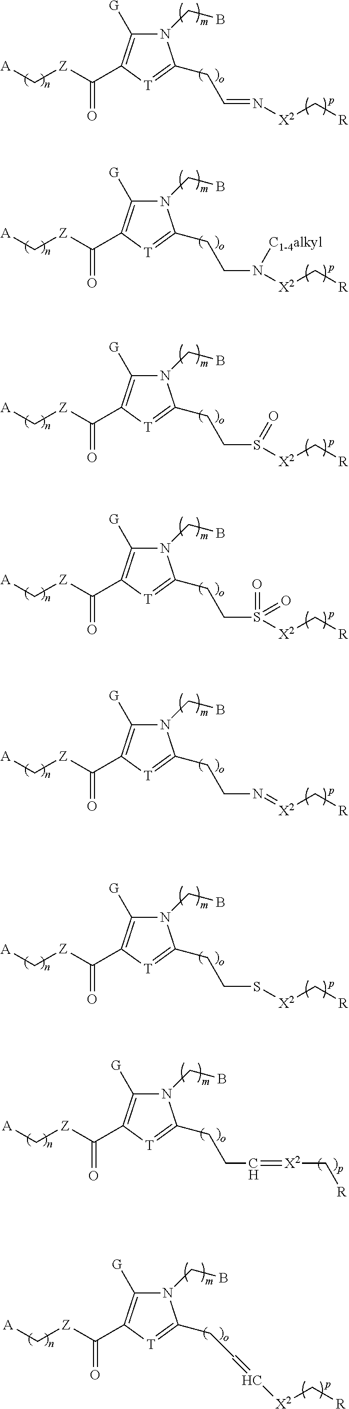

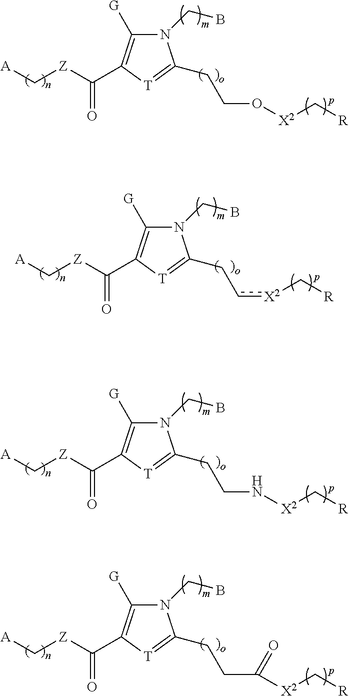

X² is a bond,

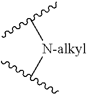

having from 1 to 4 carbon atoms,

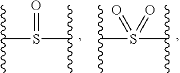

C═O, —CH═, ═CH—, NH, ═N—, —N═, S, or O. Thus, compounds having the structures below are also contemplated:

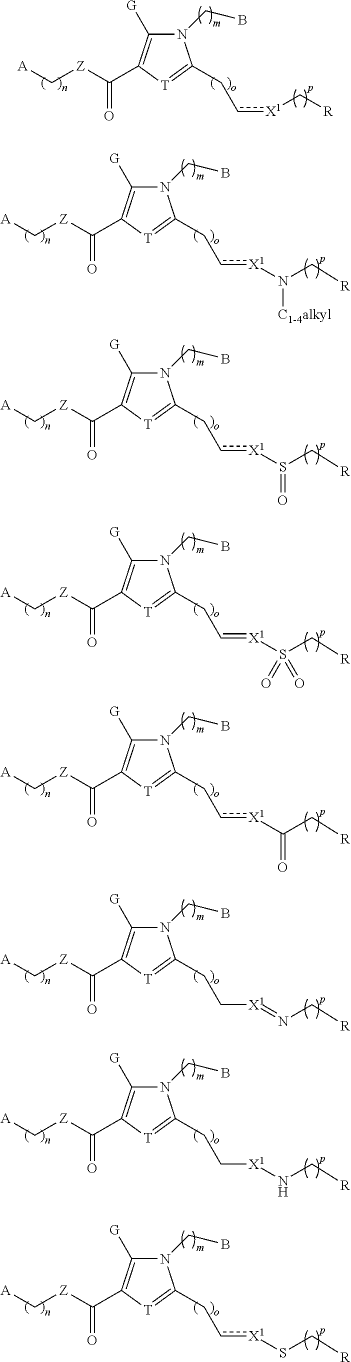

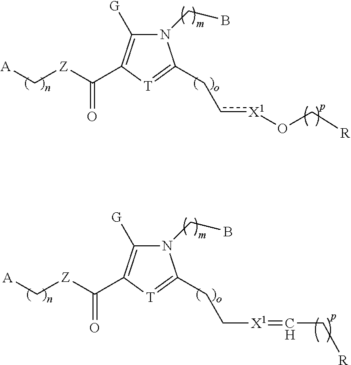

Another embodiment is a compound represented by:

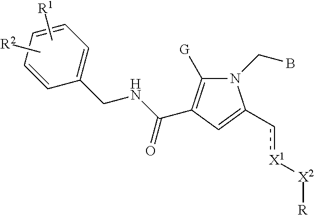

wherein R¹ and R² are independently H, F, Cl, NO₂, methyl, ethyl, n-propyl, or iso-propyl;
B is phenyl or pyridinyl which is unsubstituted, or has 1 or 2 substituents independently selected from F, Cl, NO₂, methyl, ethyl, n-propyl, and iso-propyl;
X¹ and X² are independently a bond, ═N, O, or ═CH—;
R is C₁₋₅ alkyl, or phenyl which is unsubstituted, or has 1 or 2 substituents independently selected from F, Cl, NO₂, methyl, ethyl, n-propyl, and iso-propyl.

C₁₋₅ alkyl is alkyl having 1, 2, 3, 4, or 5 carbon atoms.

In another embodiment X¹-X² are selected from ═C—, ═N—O—, and O.

In another embodiment B is unsubstituted phenyl.

In another embodiment B is unsubstituted pyridinyl.

In another embodiment R is iso-propyl.

In another embodiment R is methylphenyl. Methylphenyl is:

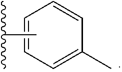

In another embodiment R is thiazolyl.

In another embodiment R is oxazolyl.

In another embodiment R is oxazolinyl.

In another embodiment R is n-butyl.

In another embodiment R¹ and R² are independently H, methyl, F, or NO₂.

In another embodiment Z is N or CH₂.

In another embodiment T is CH.

In another embodiment m is 0.

In another embodiment n is 1.

Compounds according to the teachings of the present invention include:

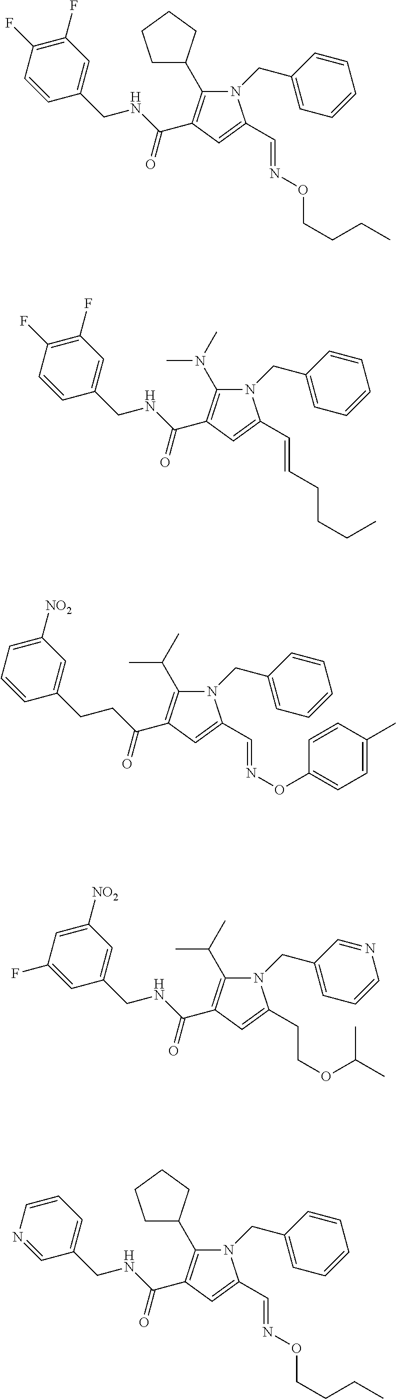

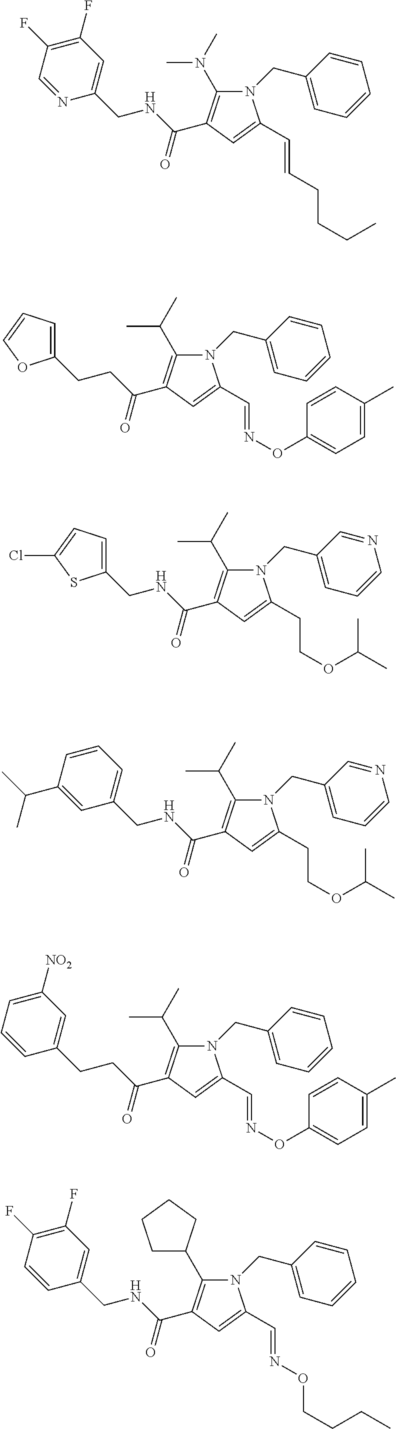

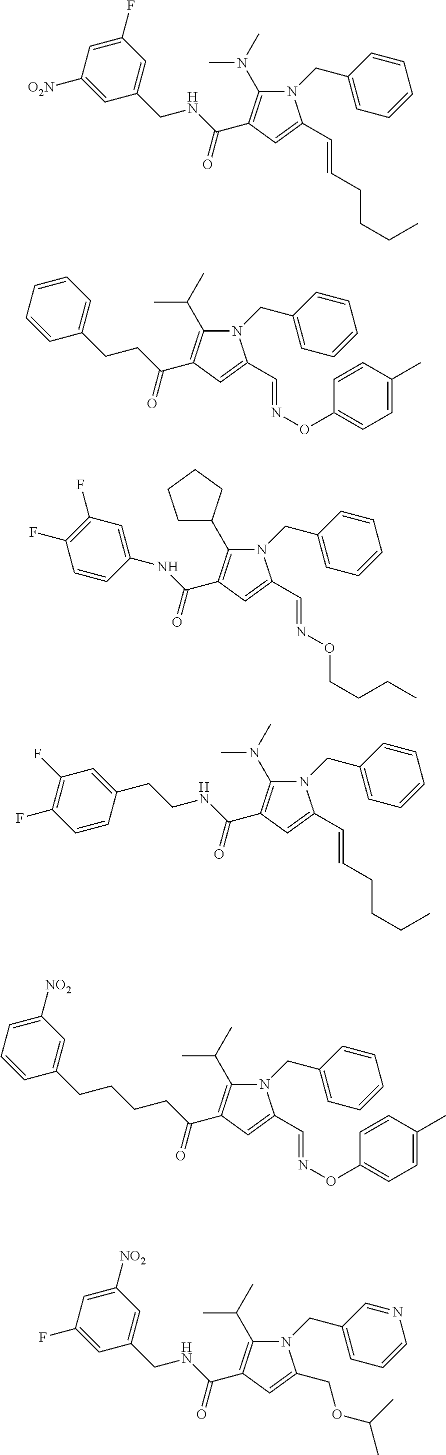

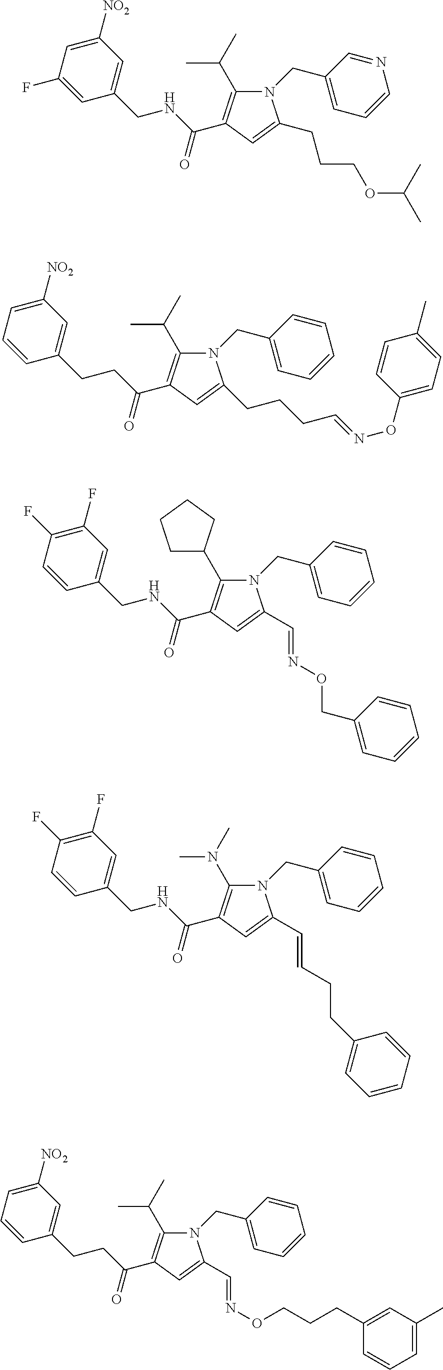

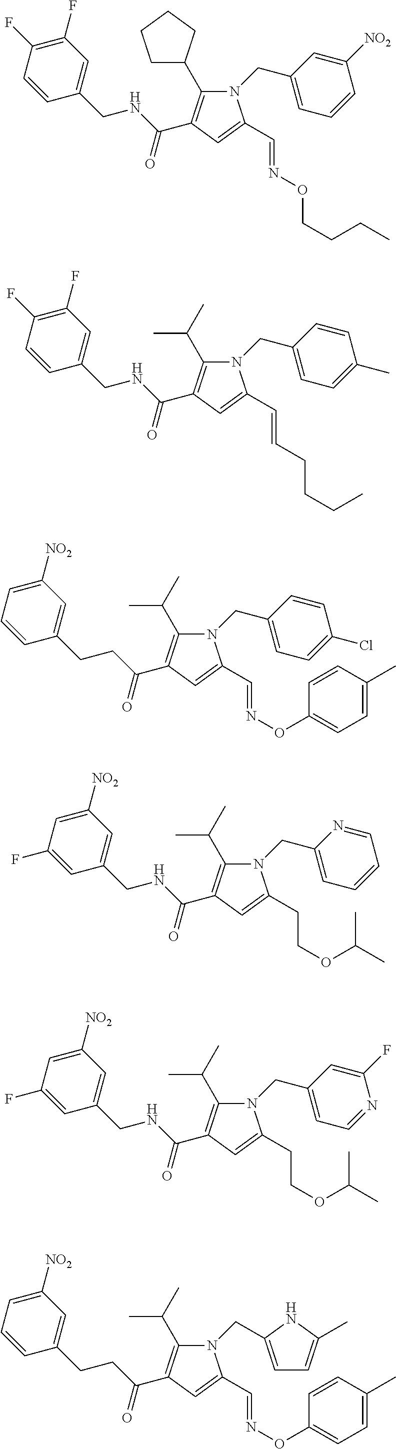

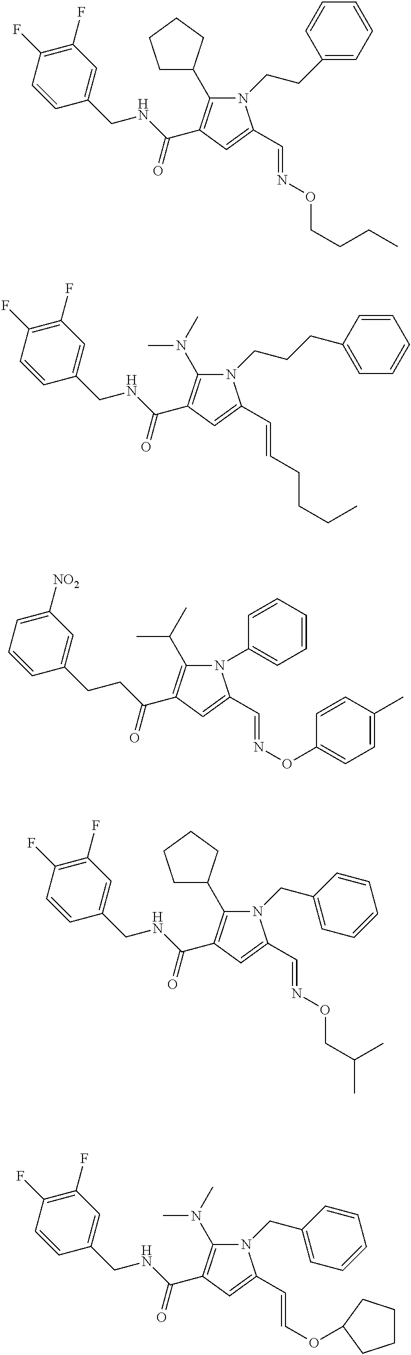

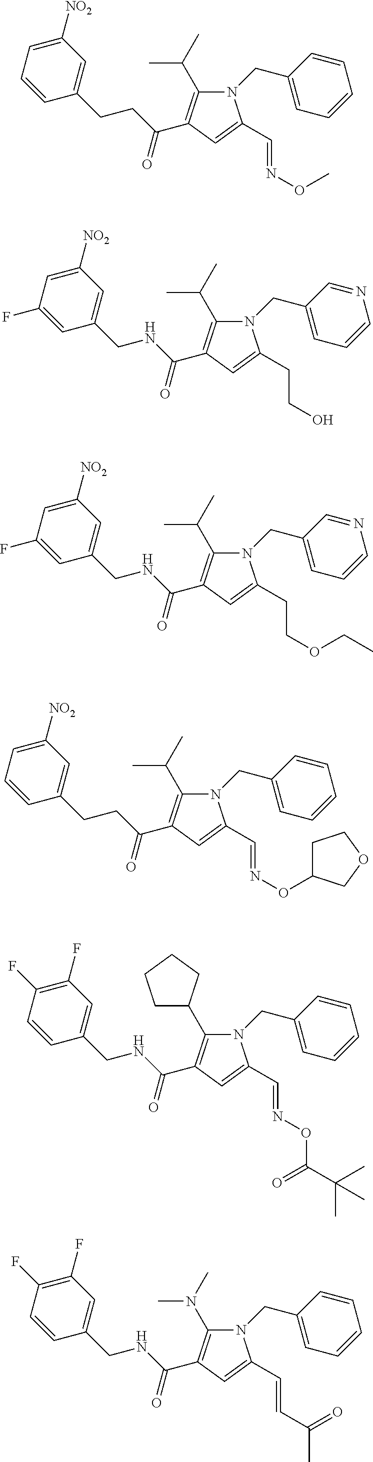

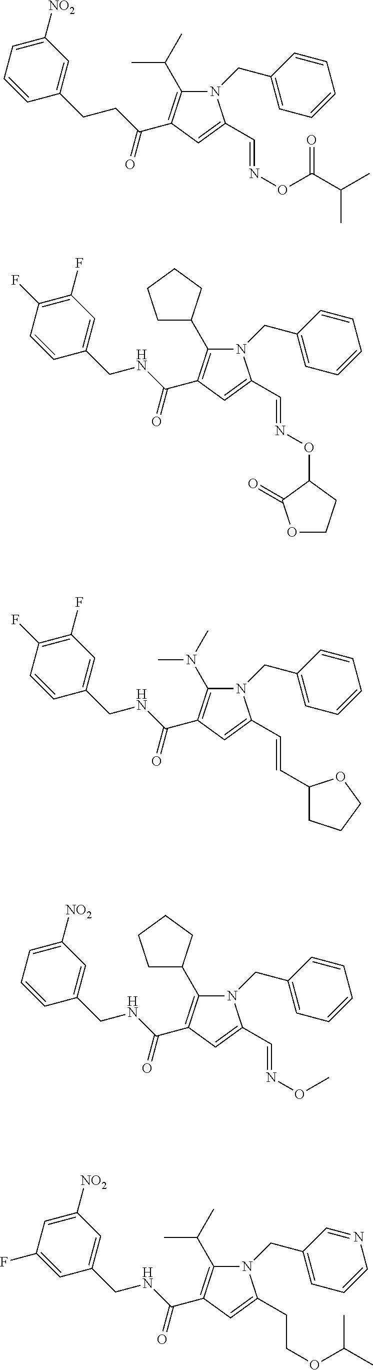

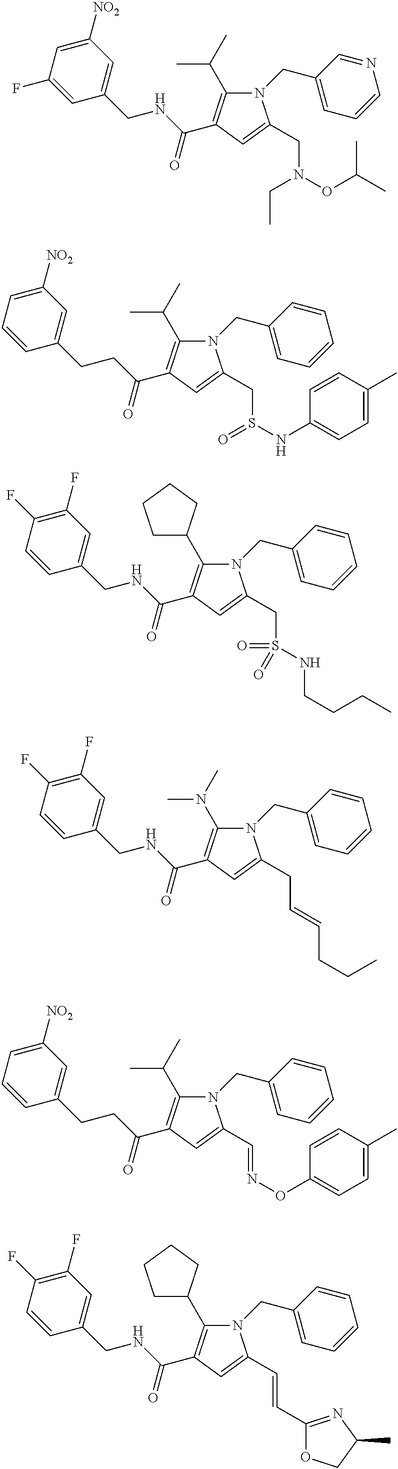

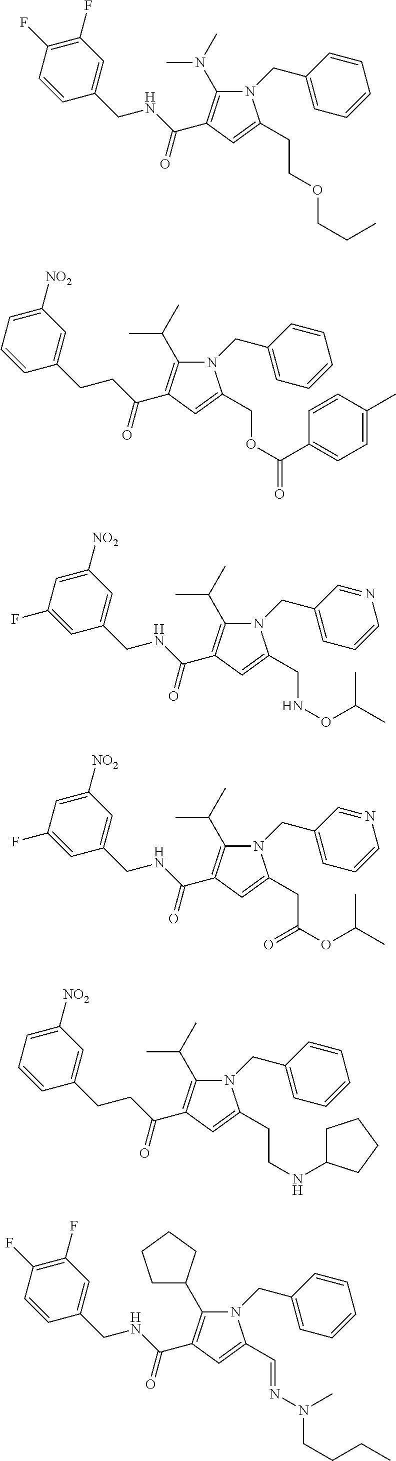

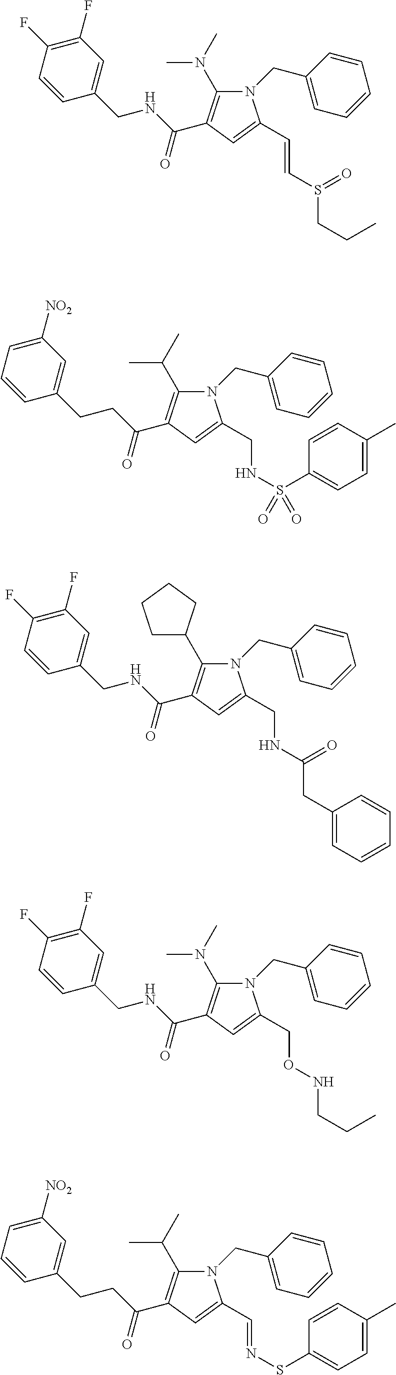

### Methods of Synthesis

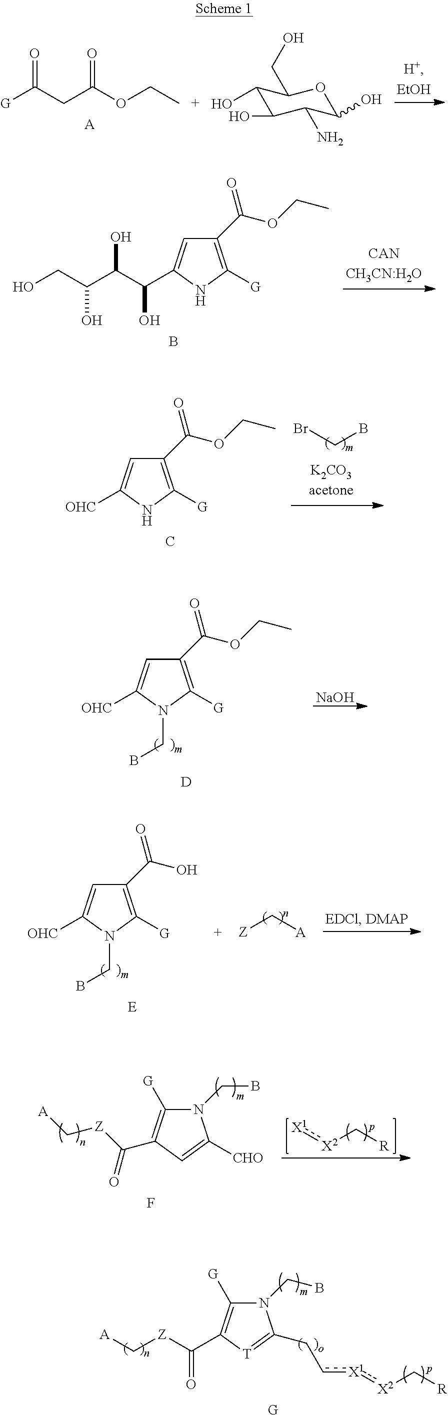

Scheme 1 illustrates one possible method for making the compounds disclosed herein where T is CH. In this method, G is provided in starting compound A. Many of these compounds are commercially available. If not, these compounds can be easily prepared from commercially available compounds. For example, ethyl malonyl chloride could be added to a dialkylcopper reagent using conventional procedures to obtain the desired compound A. Compound A is reacted with glucosamine to provide the core pyrrole in compound B. The residual polyol fragment from the glucosamine is oxidatively cleaved with a reagent such as ceric ammonium nitrate (CAN) to provide the aldehyde functionality of compound C. The linear alkyl-B fragment may be added using the corresponding alkyl halide, such as benzylbromide, and a base to form compound D. Coupling of Br—B to the nitrogen of C is accomplished by an Ullman N-arylation reaction (ref: Journal of Organic Chemistry, 72(8), 2737-2743, 2007). Compounds such as Br—(CH₂)ₘ—B are commercially available, or can be prepared by conventional methods. For example, an arylaldehyde could be reduced to the alcohol, and then converted to the corresponding alkyl halide. Longer alkyl fragments may be provided, for example, by utilizing a Wittig or a Horner-Emmons, or similar reaction, or by adapting methods described in EP637580; Journal of the American Chemical Society 107(24) 7164-7, 1985; and Journal of the American Chemical Society 106(25) 7887-90, 1984. Z-(CH₂)ₙ-A may be added by traditional substitution reactions available for carboxylic acid derivatives to provide compound F. Z-(CH₂)ₙ-A might be prepared by a number of methods. For example, the methods described above could be used to prepare Br—(CH₂)ₙ-A, which could then be modified to provide the desired functionality at Z using standard methods such as substitution. Standard methods can then be employed to add the

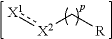

fragment to the aldehyde of compound F to give compound G.

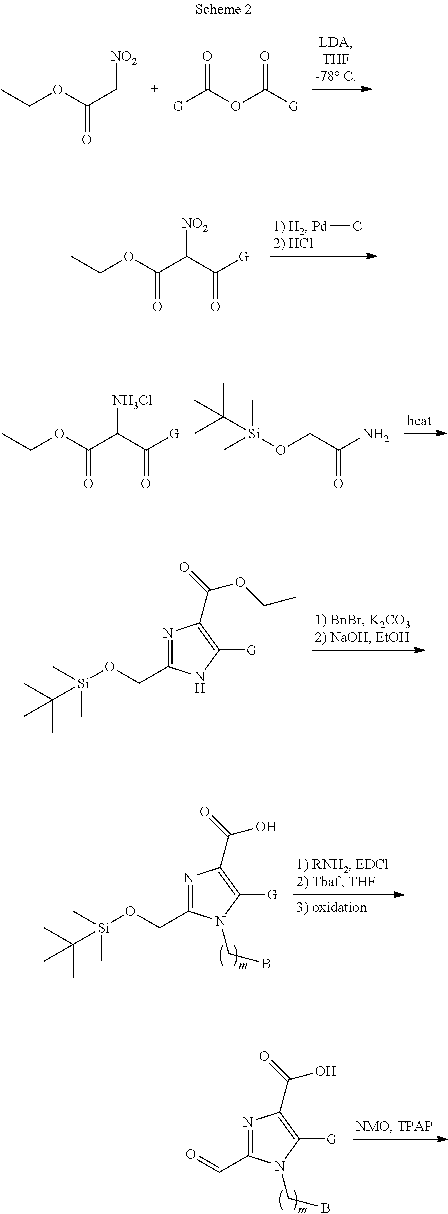

Scheme 2 illustrates another possible method of making the compounds where T is N. The product of this scheme can be substituted for compound E in scheme 1.

Two additional theoretical examples of making the compounds are depicted in Scheme 3 and Scheme 4.

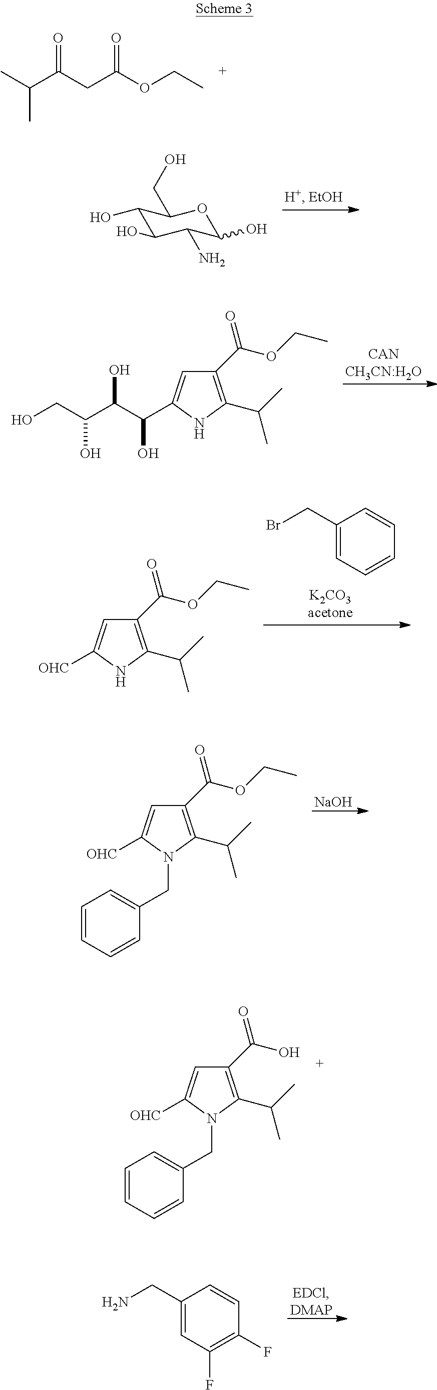

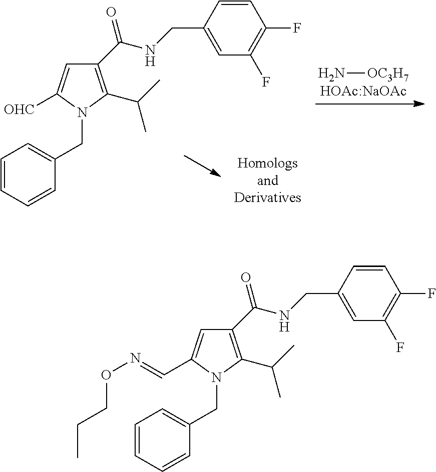

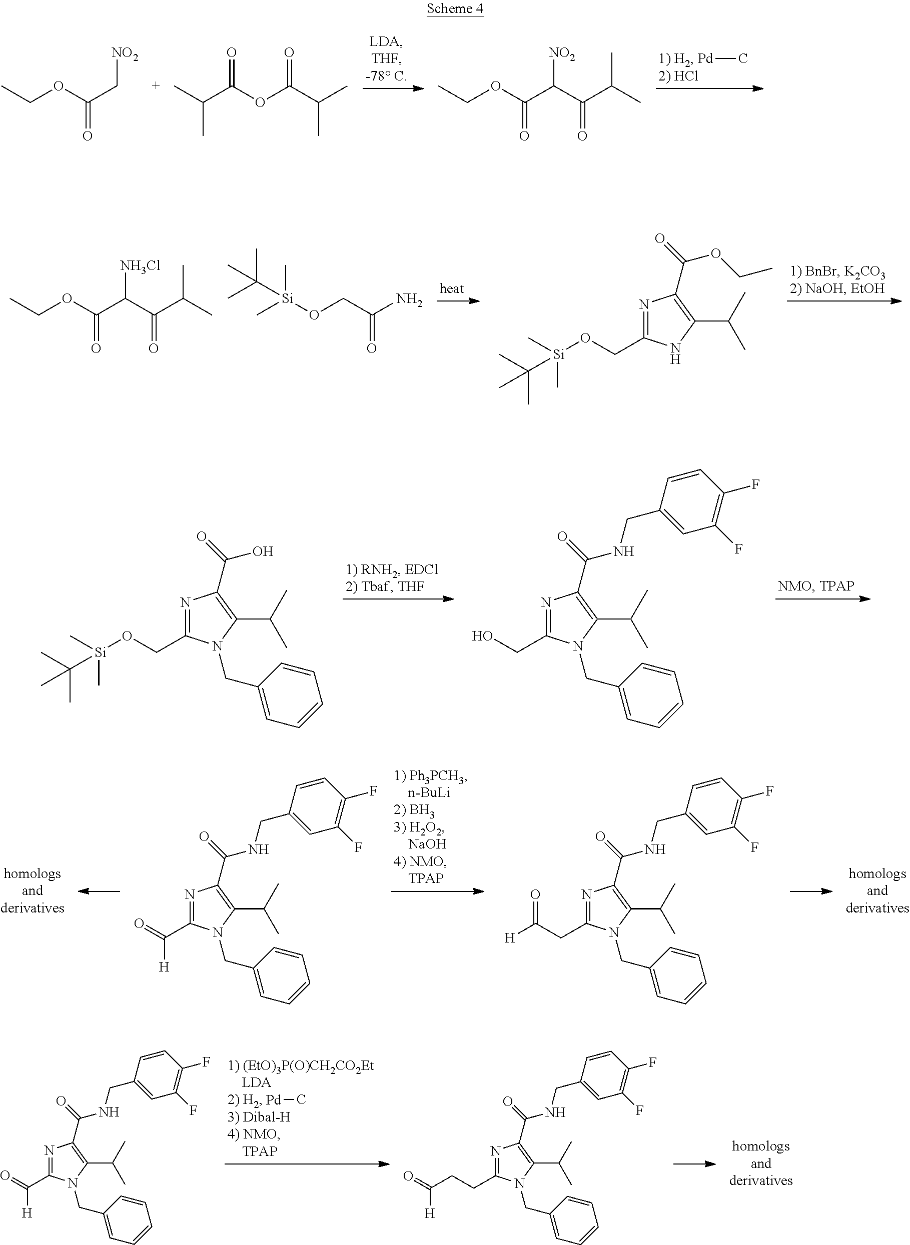

These compounds may be assessed for their ability to activate or block activation of the human S1P3 receptor in T24 cells stably expressing the human S1P3 receptor by the following procedure. Ten thousand cells/well are plated into 384-well poly-D-lysine coated plates one day prior to use. The growth media for the S1P3 receptor expressing cell line is McCoy's 5A medium supplemented with 10% charcoal-treated fetal bovine serum (FBS), 1% antibiotic-antimycotic and 400 μg/ml geneticin. On the day of the experiment, the cells are washed twice with Hank's Balanced Salt Solution supplemented with 20 mM HEPES (HBSS/Hepes buffer). The cells are then dye loaded with 2 uM Fluo-4 diluted in the HBSS/Hepes buffer with 1.25 mM Probenecid and incubated at 37° C. for 40 minutes. Extracellular dye is removed by washing the cell plates four times prior to placing the plates in the FLIPR (Fluorometric Imaging Plate Reader, Molecular Devices). Ligands are diluted in HBSS/Hepes buffer and prepared in 384-well microplates. The positive control, Sphingosine-1-Phosphate (S1P), is diluted in HBSS/Hepes buffer with 4 mg/ml fatty acid free bovine serum albumin. The FLIPR transfers 12.5 μl from the ligand microplate to the cell plate and takes fluorescent measurements for 75 seconds, taking readings every second, and then for 2.5 minutes, taking readings every 10 seconds. Drugs are tested over the concentration range of 0.61 nM to 10,000 nM. Data for Ca⁺² responses are obtained in arbitrary fluorescence units and not translated into Ca⁺² concentrations. IC₅₀ values are determined through a linear regression analysis using the Levenburg Marquardt algorithm.

### Additional Methods of Synthesis

The invention is further illustrated by the following examples which are illustrative of a specific mode of practicing the invention and are not intended as limiting the scope of the claims. Unless otherwise indicated, the following Chemical Abbreviations are used in the examples:
Ac₂O: Acetic Anhydride
n-Bu: n-butyl
Bz: benzyl
CH₃CN: acetonitrile
DCM: dichloromethane
DMAP: 4-dimethylaminopyridine
DMF: N,N-dimethylformamide
DMSO: dimethyl sulfoxide
EDCI: N-(3-dimethylaminopropyl)-N′-ethylcarbodiimide
Et: ethyl
Et₂O: diethyl ether
EtOAc: ethyl acetate
EtOH: ethanol
H₂: hydrogen
H₂O: water
H₂SO₄: sulfuric acid
HBr: hydrogen bromide
HCl: hydrochloric acid
HOAc: acetic acid
i-Pr: iso-propyl
i-PrCOCl: isobutyryl chloride
K₂CO₃: potassium carbonate
Me: methyl
MgSO₄: magnesium sulfate
N₂: nitrogen
Na₂CO₃: sodium carbonate
Na₂SO₄: sodium sulfate
NaHCO₃: sodium bicarbonate
NaOH: sodium hydroxide
NH₄Cl: ammonium chloride
i-PrCOCl: iso-butyryl chloride
Pd-C: palladium on activated carbon
PTLC: preparative thin layer chromatography
t-BuOH: tert-butanol
TEA: triethylamine
THF: tetrahydrofuran
PTLC: preparative thin layer chromatography
   Unless otherwise noted, all reagents were purchased from Aldrich Chemical Company and were used as purchased without further purification.

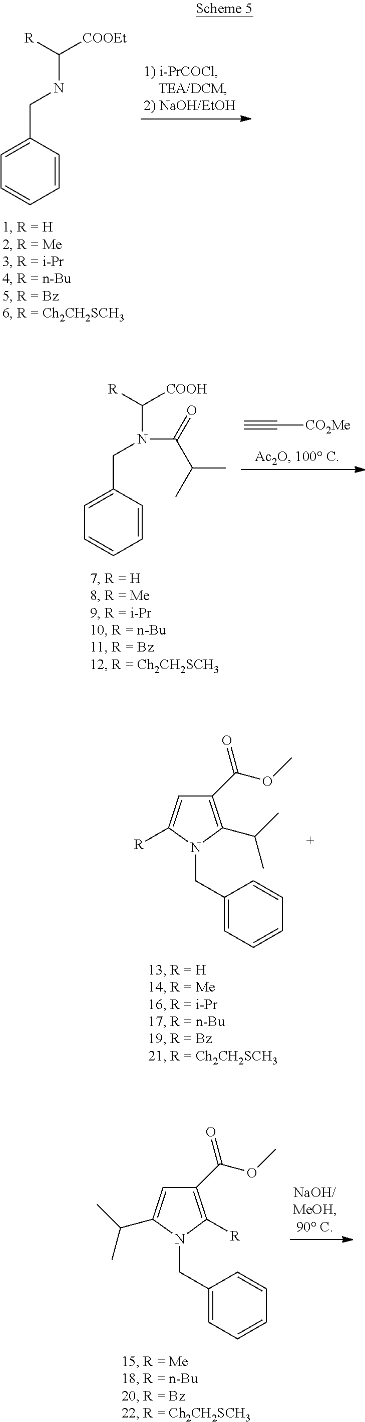

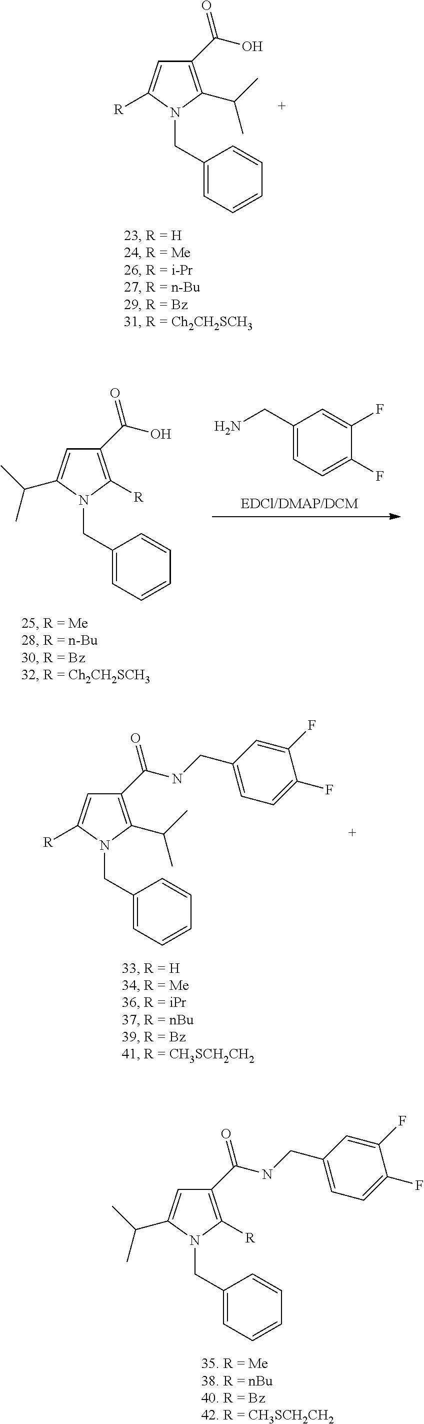

(Benzyl-isobutyryl-amino)-acetic Acid (Compound 7). General Procedure 1: Compound 7 was synthesized according to the following procedure: To N-benzyl-glycine ethyl ester (Compound 1, 5.0 g, 25.87 mmol) in 70 ml of DCM with TEA (5.4 ml, 38.8 mmol) at 0° C. was added isobutyryl chloride (3.0 g, 28.46 mmol). The reaction mixture was stirred at room temperature for 2 hours and quenched with H₂O. Two layers were separated and aqueous layer was extracted with DCM. The combined organic layers were washed with H₂O, brine, dried over Na₂SO₄ and concentrated under vacuum. Purification by column chromatography on silica gel (15% ethyl acetate in hexane) afforded 2.52 g of (benzyl-isobutyryl-amino)-acetic acid ethyl ester as oil. The ester was treated with 2N aqueous NaOH (10 ml) in EtOH (10 ml) at ROOM TEMPERATURE for 24 hours. The reaction was quenched with 6N HCl, extracted with DCM, washed with brine, dried over Na₂SO₄, and concentrated under reduced pressure to afford the title compound as colorless oil.

1H-NMR (CDCl₃): 1.18 (d, J=6.74 Hz, 1.2H), 1.19 (d, J=6.74 Hz, 4.8H), 2.68 (hept, J=6.74 Hz, 0.2H), 2.90 (hept, J=6.74 Hz, 0.8H), 4.00 (s, 0.4H), 4.06 (s, 1.6H), 4.67 (s, 2H), 7.18-7.20 (m, 2H), 7.31-7.38 (m, 3H).

Compounds 8 to 12 were also prepared by General Procedure 1:

2-(Benzyl-isobutyryl-amino)-propionic acid (Compound 8) was prepared as a white solid from N-benzyl-alanine ethyl ester (Compound 2, 3.48 g, 16.80 mmol), TEA (3.5 ml, 25.0 mmol), and isobutyryl chloride (1.97 g, 18.5 mmol).

1H-NMR (CDCl₃): 1.14 (d, J=6.74 Hz, 3H), 1.16 (d, J=6.74 Hz, 3H), 1.40 (d, J=7.33 Hz, 3H), 2.73 (hept, J=6.74 Hz, 1H), 4.50-4.60 (m, 2H), 4.60 (d, J=16.50 Hz, 1H), 7.20-7.38 (m, 5H).

2-(Benzyl-isobutyryl-amino)-3-methyl-1-butyric acid (Compound 9) was prepared as a white solid from N-benzyl-valine methyl ester (Compound 3, 2.44 g, 11 mmol), TEA (2.3 ml, 16.5 mmol), and isobutyryl chloride (1.18 g, 11.15 mmol).

1H-NMR (CDCl₃): 0.88 (d, J=6.74 Hz, 3H), 0.98 (d, J=6.74 Hz, 3H), 1.13 (d, J=6.74 Hz, 3H), 1.22 (d, J=7.33 Hz, 3H), 2.51 (m, 1H), 2.88 (hept, J=6.74 Hz, 1H), 3.54 (d, J=10.84 Hz, 1H), 4.41 (d, J=16.41 Hz, 1H), 4.83 (d, J=16.41 Hz, 1H), 7.19 (d, J=6.87 Hz, 2H), 7.26-7.38 (m, 3H).

2-(Benzyl-isobutyryl-amino)-hexanoic acid (Compound 10) was prepared as an oil from N-benzyl-L-norleucine methyl ester HCl salt (Compound 4, 3.0 g, 11.0 mmol), TEA (4 ml, 28.5 mmol), and isobutyryl chloride (1.5 g, 15.0 mmol).

1H-NMR (CDCl₃): 0.81 (d, J=6.74 Hz, 3H), 1.14 (d, J=6.74 Hz, 3H), 1.17 (d, J=6.74 Hz, 3H), 1.17-1.25 (m, 4H), 1.67-1.77 (m, 1H), 2.01-2.11 (m, 1H), 2.70 (hept, J=6.74 Hz, 1H), 4.25-4.30 (m, 1H), 4.51 (d, J=17.00 Hz, 1H), 4.70 (d, J=17.00 Hz, 1H), 7.18-7.38 (m, 5H).

2-(Benzyl-isobutyryl-amino)-3-phenyl-1-propionic acid (Compound 11) was prepared as a white solid from N-benzyl-phenylalanine methyl ester HCl salt (Compound 5, 3.05 g, 10.0 mmol), TEA (3.5 ml, 25.0 mmol), and isobutyryl chloride (1.17 g, 11.0 mmol).

1H-NMR (CDCl₃): 1.10 (d, J=6.74 Hz, 3H), 1.16 (d, J=6.74 Hz, 3H), 2.70 (hept, J=6.74 Hz, 1H), 3.34-3.38 (m, 2H), 3.73 (d, J=16.70 Hz, 1H), 4.10-4.15 (m, H), 4.46 (d, J=16.70 Hz, 1H), 7.06-7.15 (m, 4H), 7.20-7.32 (m, 6H).

2-Benzylamino-4-methylsulfanyl-butyric acid (Compound 12) was prepared as a solid from N-benzyl-methionine methyl ester HCl salt (5.0 g, 17.25 mmol), TEA (7.26 ml, 51.75 mmol), and isobutyryl chloride (Compound 6, 2.39 g, 22.4 mmol).

1H-NMR (CDCl₃): 1.17-1.25 (m, 6H), 1.98 (m, 3H), 2.00-2.10 (m, 1H), 2.38-2.50 (m, 3H), 2.85 (hept, J=6.74 Hz, 1H), 4.11-4.15 (m, 1H), 4.56 (d, J=16.87 Hz, 1H), 4.72 (d, J=16.87 Hz, 1H), 7.24 -7.41 (m, 5H).

1-Benzyl-2-isopropyl-1H-pyrrole-3-carboxylic Acid Methyl Ester (Compound 13). General Procedure 2: Compound 13 was made according to the following procedure: A mixture of (benzyl-isobutyryl-amino)-acetic acid (Compound 7, 2.18 g, 9.27 mmol), acetic anhydride (10 ml) and methyl propiolate (3.5 g, 41.6 mmol) was stirred at 100° C. for 3 hours. The solution was cooled to room temperature and the excess of acetic anhydride was removed under vacuum. The product was extracted with ether, washed with H₂O, brine, dried over Na₂SO₄ and concentrated. The title product was isolated as a major product by column chromatography on silica gel (5% ethyl acetate in hexane).

1H-NMR (CDCl₃): 1.25 (d, J=7.00 Hz, 6H), 3.48 (hept, J=7.00 Hz, 1H), 3.79 (s, 3H), 5.14 (s, 2H), 6.47 (d, J=2.93 Hz, 1H), 6.60 (d, J=2.93 Hz, 1H), 6.97-7.00 (m, 2H), 7.27-7.35 (m, 3H).

Compounds 14 to 22 were also prepared by General Procedure 2:

1-Benzyl-2-isopropyl-5-methyl-1H-pyrrole-3-carboxylic acid methyl ester (Compound 14) and 1-benzyl-5-isopropyl-2-methyl-1H-pyrrole-3-carboxylic acid methyl ester (Compound 15) were prepared from (2-benzyl-isobutyryl-amino)-propionic acid (Compound 8, 1.27 g, 5.74 mmol), acetic anhydride (8 ml) and methyl propiolate (2.17 g, 25.83 mmol). The two compounds were separated by column chromatography on silica gel.

1-Benzyl-2-isopropyl-5-methyl-1H-pyrrole-3-carboxylic Acid Methyl Ester (Compound 14):

1H-NMR (CDCl₃): 1.25 (d, J=7.03 Hz, 6H), 2.07 (s, 3H), 3.48 (m, 1H), 3.78 (s, 3H), 5.13 (s, 2H), 6.36 (s, 1H), 6.87 (d, J=6.87 Hz, 2H), 7.27-7.38 (m, 3H).

1-Benzyl-5-isopropyl-2-methyl-1H-pyrrole-3-carboxylic Acid Methyl Ester (Compound 15):

1H-NMR (CDCl₃): 1.17 (d, J=6.74 Hz, 6H), 2.42 (s, 3H), 2.73 (hept, J=6.74 Hz, 1H), 3.80 (s, 3H), 5.09 (s, 2H), 6.38 (s, 1H), 6.85 (d, J=6.87 Hz, 2H), 7.22-7.35 (m, 3H).

1-Benzyl-2, 5-diisopropyl-1H-pyrrole-3-carboxylic acid methyl ester (Compound 16) was prepared as oil from (2-benzyl-isobutyryl-amino)-3-methyl-1-butyric acid (Compound 9, 1.51 g, 5.45 mmol), acetic anhydride (8 ml) and methyl propiolate (2.06 g, 24.5 mmol).

1H-NMR (CDCl₃): 1.17 (d, J=6.74 Hz, 6H), 1.24 (d, J=7.33 Hz, 6H), 2.68 (hept, J=6.73 Hz, 1H), 3.35 (m, 1H), 3.79 (s, 3H), 5.16 (s, 2 H), 6.42 (s, 1H), 6.86 (d, J=6.84 Hz, 2H), 7.20-7.32 (m, 3H).

1-Benzyl-5-butyl-2-isopropyl-1H-pyrrole-3-carboxylic acid methyl ester (Compound 17) and 1-benzyl-2-butyl-5-isopropyl-1H-pyrrole-3-carboxylic acid methyl ester (Compound 18) were prepared as an inseparable mixture from (2-benzyl-isobutyryl-amino)-hexanoic acid (Compound 10, 1.02 g, 3.50 mmol), acetic anhydride (8 ml) and methyl propiolate (1.26 g, 15.0 mmol), and the mixture was used in the next reaction after purification by silica gel chromatography.

1,5-Dibenzyl-2-isopropyl-1H-pyrrole-3-carboxylic acid methyl ester (Compound 19) and 1, 2-dibenzyl-5-isopropyl-1H-pyrrole-3-carboxylic acid methyl ester (Compound 20) were prepared as an inseparable mixture from (2-benzyl-isobutyryl-amino)-3-phenyl-1-propionic acid (Compound 11, 1.07 g, 3.31 mmol), acetic anhydride (8 ml) and methyl propiolate (1.26 g, 15.0 mmol), and the mixture was used in the next reaction after purification by silica gel chromatography.

1-Benzyl-2-isopropyl-5-(2-methylsuffanyl-ethyl)-1H-pyrrole-3-carboxylic acid methyl ester (Compound 21) and 1-benzyl-5-isopropyl-2-(2-methylsulfanyl-ethyl)-1H-pyrrole-3-carboxylic acid methyl ester (Compound 22) were prepared as an inseparable mixture from 2-benzylamino-4-methylsulfanyl-butyric acid (Compound 12, 2.2 g, 7.12 mmol), acetic anhydride (8 ml) and methyl propiolate (2.39 g, 28.48 mmol), and the mixture was used in the next reaction after purification by silica gel chromatography.

1-Benzyl-2-isopropyl-1H-pyrrole-3-carboxylic Acid (Compound 23). General Procedure 3: Compound 23 was prepared according to the following procedure: 1-Benzyl-2-isopropyl-1H-pyrrole-3-carboxylic acid methyl ester (Compound 13, 550 mg, 2.14 mmol) was treated with 5N aqueous NaOH (1 ml) in MeOH (10 ml ) at 80° C. for 24 hours. The reaction solution was cooled to RT and neutralized with 10% aqueous HCl to precipitate out the title product as while solid.

1H-NMR (CDCl₃): 1.27 (d, J=7.33 Hz, 6H), 3.53 (hept, J=7.33 Hz, 1H), 5.16 (s, 2H), 6.48 (d, J=2.93 Hz, 1H), 6.69 (d, J=2.93 Hz, 1H), 6.99-7.02 (m, 2H), 7.25-7.36 (m, 3H).

Compounds 24 to 32 were also prepared by General Procedure 3:

1-Benzyl-2-isopropyl-5-methyl-1 H-pyrrole-3-carboxylic acid (Compound 24) was prepared as a white solid from 1-benzyl-2-isopropyl-5-methyl-1H-pyrrole-3-carboxylic acid methyl ester (compound 14, 175 mg, 0.65 mmol) and 5N NaOH.

1H-NMR (CDCl₃): 1.26 (d, J=7.33 Hz, 6H), 2.08 (s, 3H), 3.55 (m, 1H), 5.13 (s, 2H), 6.44 (s, 1H), 6.89 (d, J=6.87 Hz, 2H), 7.24-7.34 (m, 3H).

1-Benzyl-5-isopropyl-2-methyl-1H-pyrrole-3-carboxylic acid (Compound 25) was prepared as a white solid from 1-benzyl-5-isopropyl-2-methyl-1H-pyrrole-3-carboxylic acid methyl ester (Compound 8, 475 mg, 1.75 mmol) and 5N NaOH.

1H-NMR (CDCl₃): 1.18 (d, J=6.74 Hz, 6H), 2.43 (s, 3H), 2.73 (hept, J=6.74 Hz, 1H), 5.10 (s, 2H), 6.45 (s, 1H), 6.87 (d, J=6.87 Hz, 2H), 7.22-7.35 (m, 3H).

1-Benzyl-2, 5-diisopropyl-1H-pyrrole-3-carboxylic acid (Compound 26) was prepared as a white solid from 1-benzyl-2,5-diisopropyl-1H-pyrrole-3-carboxylic acid methyl ester (Compound 16, 1000 mg, 3.34 mmol) and 5N NaOH.

1H-NMR (CDCl₃): 1.17 (d, J=6.74 Hz, 6H), 1.25 (d, J=7.03 Hz, 6H), 2.69 (hept, J=7.03 Hz, 1H), 3.38 (m, 1H), 5.18 (s, 2H), 6.50 (s, 1H), 6.87 (d, J=6.84 Hz, 2H), 7.22-7.33 (m, 3H).

1-Benzyl-5-butyl-2-isopropyl-1H-pyrrole-3-carboxylic acid (Compound 27) and 1-benzyl-2-butyl-5-isopropyl-1H-pyrrole-3-carboxylic acid (Compound 28) were prepared as an oil from a mixture of 1-benzyl-5-butyl-2-isopropyl-1H-pyrrole-3-carboxylic acid methyl ester and 1-benzl-2-butyl-5-isopropyl-1 H-pyrrole-3-carboxylic acid methyl ester (Compounds 17 and 18, respectively, 900 mg, 2.87 mmol) and 5N NaOH), and the mixture was used in the next reaction without further purification.

1,5-Dibenzyl-2-isopropyl-1H-pyrrole-3-carboxylic acid (Compound 29) and 1,2-dibenzyl-5-isopropyl-1H-pyrrole-3-carboxylic acid (Compound 30) were prepared as a white solid from a mixture of 1,5-dibenzyl-2-isopropyl-1H-pyrrole-3-carboxylic acid methyl ester and 1,2-dibenzyl-5-isopropyl-1H-pyrrole-3-carboxylic acid methyl ester (Compounds 19 and 20, respectively, 1.1 g, 3.17 mmol) and 5N NaOH, and the mixture was used in the next reaction without further purification.

1-Benzyl-2-isopropyl-5-(2-methylsulfanyl-ethyl)-1H-pyrrole-3-carboxylic acid (Compound 31) and 1-benzyl-5-isopropyl-2-(2-methylsulfanyl-ethyl)-1H-pyrrole-3-carboxylic acid (Compound 32) were prepared as an oil from a mixture of 1-benzyl-2-isopropyl-5-(2-methylsulfanyl-ethyl)-1H-pyrrole-3-carboxylic acid methyl ester and 1-benzyl-5-isopropyl-2-(2-methylsulfanyl-ethyl)-1H-pyrrole-3-carboxylic acid methyl ester (Compounds 21 and 22, respectively, 450 mg, 1.36 mmol) and 5N NaOH, and the mixture was used in the next reaction without further purification.

1-Benzyl-2-isopropyl-1H-pyrrole-3-carboxylic Acid 3,4-Difluoro-benzylamide (Compound 33). General Procedure 4: Compound 33 was prepared according to the following procedure: To a solution of 1-benzyl-2-isopropyl-1H-pyrrole-3-carboxylic acid (Compound 23, 310 mg, 1.27 mmol) in CH₂Cl₂ (20 ml ) and DMF (4 ml ) was added EDCI (315 mg, 1.65 mmol), DMAP (232 mg, 1.90 mmol) and 3,4-difluoro-benzylamine (182 mg, 1.27 mmol). The mixture was stirred at room temperature for 16 h, diluted with DCM, and washed with aqueous NaHCO₃, and brine, and dried over Na₂SO₄, and concentrated under reduced pressure. The residue was purified by flash column chromatography on silica gel (10% to 15% ethyl acetate in hexanes) to yield the title compound as a beige solid.

1H-NMR (CDCl₃): 1.29 (d, J=7.33 Hz, 6H), 3.55 (hept, J=7.33 Hz, 1H), 4.52 (d, J=5.28 Hz, 2H), 5.14 (s, 2H), 6.15 (bs, 1H), 6.26 (d, J=2.93 Hz, 1H), 6.47 (d, J=2.93Hz, 1H), 6.99-7.36 (m, 8H).

Compounds 34 to 42 were also prepared by General Procedure 4:

1-Benzyl-2-isopropyl-5-methyl-1H-pyrrole-3-carboxylic Acid 3,4-Difluoro-benzylamide (Compound 34) was prepared as a white solid from 1-benzyl-2-isopropyl-5-methyl-1H-pyrrole-3-carboxylic acid (150 mg, 0.58 mmol), EDCI (144 mg, 0.75 mmol), DMAP (106 mg, 0.87 mmol), and 3,4-difluorobenzylamine (100 mg, 0.70 mmol).

1H-NMR (CDCl₃): 1.28 (d, J=7.03 Hz, 6H), 2.06 (s, 3H), 3.59 (hept, J=7.03 Hz, 1H), 4.52 (d, J=5.57 Hz, 2H), 5.13 (s, 2H), 6.03 (s, 1H), 6.05 (bs, 1H), 6.80 (d, J=6.87Hz, 2H), 7.04-7.36 (m, 6H).

1-Benzyl-5-isopropyl-2-methyl-1 H-pyrrole-3-carboxylic acid 3,4-difluoro-benzylamide (Compound 35) was prepared) as a beige solid from 1-benzyl-5-isopropyl-2-methyl-1H-pyrrole-3-carboxylic acid (Compound 25, 310 mg, 1.21 mmol), EDCI (300 mg, 1.57 mmol), DMAP (222 mg, 1.82mmol), and 3,4-difluorobenzylamine (108mg, 1.45mmol).

1H-NMR (CDCl₃): 1.15 (d, J=7.03 Hz, 6H), 2.06 (s, 3H), 2.75 (hept, J=7.03 Hz, 1H), 4.54 (d, J=5.57 Hz, 2H), 5.09 (s, 2 H), 6.05 (s, 1H), 6.07 (bs, 1H), 6.85 (d, J=6.87 Hz, 2H), 7.08-7.32 (m, 6H).

1-Benzyl-2,5-diisopropyl-1H-pyrrole-3-carboxylic acid 3,4-diflurobenzylamine (Compound 36) was prepared as a white solid from 1-benzyl-2,5-diisopropyl-1H-pyrrole-3-carboxylic acid (Compound 26, 368 mg, 1.29 mmol), EDCI (320 mg, 1.68 mmol), DMAP (237 mg, 1.94 mmol), and 3,4-difluorobenzylamine (221 mg, 1.55 mmol).

1H-NMR (CDCl₃): 1.17 (d, J=6.74 Hz, 6H), 1.27 (d, J=7.03 Hz, 6H), 2.68 (hept, J=6.74Hz, 1H), 3.41 (hept, J=7.03 Hz, 1H), 4.54 (d, J=5.57 Hz, 2H), 5.16 (s, 2 H), 6.04 (s, 1H), 6.06 (bs, 1H), 6.86 (d, J=6.87 Hz, 2H), 7.07-7.36 (m, 6H).

1-Benzyl-5-butyl-2-isopropyl-1H-pyrrole-3-carboxylic acid 3,4-difluoro-benzylamide (Compound 37) and 1-benzyl-2-butyl-5-isopropyl-1H-pyrrole-3-carboxylic acid 3,4-difluoro-benzylamide (Compound 38) were prepared from the mixture of 1-benzyl-5-butyl-2-isopropyl-1H-pyrrole-3-carboxylic acid and 1-benzyl-2-butyl-5-isopropyl-1H-pyrrole-3-carboxylic acid (Compounds 27 and 28, respectively, 850 mg, 2.83 mmol), EDCI (760 mg, 4.0 mmol), DMAP (614mg, 5.0mmol), and 3,4-difluorobenzylamine (487mg, 3.4mmol), and then separated by column chromatography followed by crystallization.

1-Benzyl-5-butyl-2-isopropyl-1H-pyrrole-3-carboxylic Acid 3,4-Difluoro-benzylamide (Compound 37):

1H-NMR (CDCl₃): 0.85 (t, J=7.33 Hz, 3H), 1.27 (d, J=7.33 Hz, 6H), 1.33 (m, 2H),1.51 (m, 2H), 2.35 (t, J=7.62 Hz, 2H), 3.50 (m, 1H), 4.54 (bs, 2H), 5.13 (s, 2 H), 6.02 (s, 1H), 6.03 (bs, 1H), 6.86 (d, J=6.87 Hz, 2H), 7.05-7.35 (m, 6H).

1-Benzyl-2-butyl-5-isopropyl-1H-pyrrole-3-carboxylic Acid 3,4-Difluoro-benzylamide (Compound 38):

1H-NMR (CDCl₃): 0.83 (t, J-7.33 Hz, 3H), 1.14 (d, J=6.74 Hz, 6H), 1.22-1.45 (m, 4H), 2.70 (hept, J=6.74 Hz, 1H), 2.89 (t, J=7.33 Hz, 2H), 4.56 (d, J=5.30 Hz, 2H), 5.10 (s, 2 H), 6.05 (s, 1H), 6.07 (bs, 1H), 6.86 (d, J=6.87 Hz, 2H), 7.05-7.35 (m, 6H).

1,5-dibenzyl-2-isopropyl-1H-pyrrole-3-carboxylic acid 3,4-difluoro-benzylamide (Compound 39) and 1,2-dibenzyl-5-isopropyl-1H-pyrrole-3-carboxylic acid 3,4-difluoro-benzylamide (Compound 40) were prepared from the mixture of 1,5-dibenzyl-2-isopropyl-1-H-pyrrole-3-carboxylic acid and 1,2-dibenzyl-5-isopropyl-1 H-pyrrole-3-carboxylic acid (Compounds 29 and 30, respectively, 684 mg, 2.05 mmol), EDCI (572 mg, 3.0 mmol), DMAP (427mg, 3.5 mmol), and 3,4-difluorobenzylamine (353 mg, 2.46 mmol), and then separated by HPLC.

1,5-Dibenzyl-2-isopropyl-1H-pyrrole-3-carboxylic Acid 3,4-Difluoro-benzylamide (Compound 39):

1H-NMR (CDCl₃): 1.28 (d, J=7.33 Hz, 6H), 3.56 (m, 1H), 3.69 (s, 2H), 4.51 (bs, 2H), 5.06 (s, 2H), 5.92 (s, 1H), 6.05 (bs, 1H), 6.85 (d, J=6.87 Hz, 2H), 7.05-7.35 (m, 11H).

1,2-Dibenzyl-5-isopropyl-1H-pyrrole-3-carboxylic Acid 3,4-Difluoro-benzylamide (Compound 40):

1H-NMR (CDCl₃): 1.14 (d, J=6.74 Hz, 6H), 2.70 (m, 1H), 4.33 (s, 2H), 4.54 (bs, 2H), 4.94 (s, 2H), 6.10 (bs, 1H), 6.14 (s, 1H), 6.79 (d, J=6.87 Hz, 2H), 7.06-7.32 (m, 11H).

2-Benzylamino-4-methylsulfanyl-butyric acid was prepared from N-benzyl-methionine methyl ester HCl salt (5.0 g, 17.25 mmol), TEA (7.26 ml, 51.75mmol), and isobutyryl chloride (2.39 g, 22.4 mmol) according to general procedure 1 as solid.

1H-NMR (CDCl₃): 1.17-1.25 (m, 6H), 1.98 (m, 3H), 2.00-2.10 (m, 1H), 2.38-2.50 (m, 3H), 2.85 (hept, J=6.74 Hz, 1H), 4.11-4.15 (m, 1H), 4.56 (d, J=16.87 Hz, 1H), 4.72 (d, J=16.87 Hz, 1H), 7.24-7.41 (m, 5H).

1-Benzyl-2-isopropyl-5-(2-methylsulfanyl-ethyl)-1H-pyrrole-3-carboxylic acid 3, 4-difluoro-benzylamide (Compound 41) and 1-benzyl-5-isopropyl-2-(2-methylsulfanyl-ethyl)-1H-pyrrole-3-carboxylic acid 3,4-difluoro-benzylamide (Compound 42) were prepared from the mixture of 1-benzyl-2-isopropyl-5-(2-methylsulfanyl-ethyl)-1H-pyrrole-3-carboxylic acid and 1-benzyl-5-isopropyl-2-(2-methylsulfanyl-ethyl)-1H-pyrrole-3-carboxylic acid (Compounds 31 and 32, respectively, 400 mg, 1.26 mmol), EDCI (312 mg, 1.64 mmol), DMAP (230 mg, 1.89 mmol), and 3,4-difluorobenzylamine (216 mg, 1.51 mmol), and then separated by column chromatography followed by crystallization.

1-Benzyl-2-isopropyl-5-(2-methylsulfanyl-ethyl)-1H-pyrrole-3-carboxylic Acid 3,4-Difluoro-benzylamide (Compound 41)

1H-NMR (CDCl₃): 1.28 (d, J=7.33 Hz, 6H), 2.00 (s, 3H), 2.60-2.70 (m, 4H), 3.51 (m, 1H), 4.54 (b, J=5.67 Hz, 2H), 5.16 (s, 2H), 6.09 (s, 1H), 6.10 (bs, 1H), 6.87 (d, J=6.87 Hz, 2H), 7.05-7.35 (m, 6H).

1-Benzyl-5-isopropyl-2-(2-methylsulfanyl-ethyl)-1H-pyrrole-3-carboxylic Acid 3,4-Difluoro-benzylamide (compound 42)

1H-NMR (CDCl₃): 1.16 (d, J=6.74 Hz, 6H), 2.00 (s, 3H), 2.61 (t, J=7.33 Hz, 2H), 2.74 (m, 1H), 3.16 (t, J=7.33 Hz, 2H), 4.52 (b, J=5.67 Hz, 2H), 5.18 (s, 2H), 6.08 (s, 1H), 6.18 (bs, 1H), 6.85 (d, J=6.87 Hz, 2H), 7.05-7.35 (m, 6H).

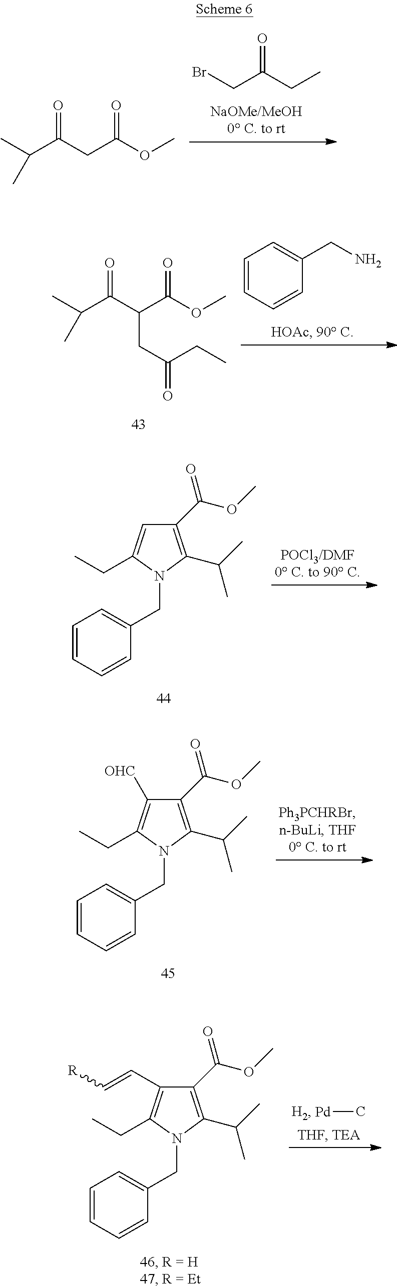

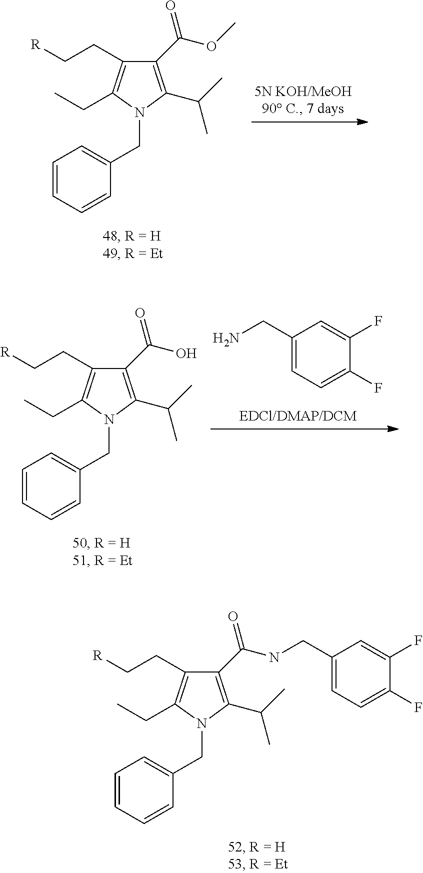

2-Isobutyryl-4-oxo-hexanoic Acid Methyl Ester (Compound 43): To a solution of NaOMe (1.3 g, 24.1 mmol) and 20 ml of anhydrous MeOH was added 4-methyl-3-oxo-pentanoic acid methyl ester (2.88 g, 20 mmol). The reaction solution was stirred at room temperature for 40 mins. 1-Bromo-2-butanone was added dropwise. The resulting solution was stirred at room temperature for 18 hours and quenched with H₂O, extracted with ether, washed with brine, dried over Na₂SO₄, and concentrated under reduced pressure. The title product was purified by column chromatography on silica gel with 5% EtOAc/Hex as oil

1H-NMR (CDCl₃): 1.04 (t, J=7.33 Hz, 3H), 1.11 (d, J=7.03 Hz, 3H), 1.17 (d, J=7.03 Hz, 3H), 2.47 (q, J=7.33 Hz, 2H), 2.87-2.97 (m, 2H), 3.08 (dd, J=7.91 and 8.21 Hz, 1H), 3.32 (s, 3H), 4.22 (dd, J=6.87 and 5.86 Hz , 2H).

1-Benzyl-5-ethyl-2-isopropyl-1H-pyrrole-3-carboxylic Acid Methyl Ester (Compound 44): To solution of 2-isobutyryl-4-oxo-hexanoic acid methyl ester (Compound 43, 389 mg, 1.82 mmol) in 2 ml of HOAc was added benzylamine (645 mg, 6.03 mmol). Stirred at 100° C. for 2 hours and cooled to room temperature. The reaction was quenched with H₂O, extracted with DCM, washed with brine, dried over Na₂SO₄, and concentrated under reduced pressure. The title product was purified by column chromatography on silica gel with 2 to 4% EtOAc/Hex as oil

1H-NMR (CDCl₃): 1.20 (t, J=7.33 Hz, 3H), 1.25 (d, J=7.33 Hz, 6H), 2.37 (q, J=7.33 Hz, 2H), 3.45 (m, 1H), 3.79 (s, 3H), 5.13 (s, 2H), 6.40 (s, 1H), 6.86 (d, J=7.13 Hz, 2H), 7.21-7.35 (m, 3H).

1-Benzyl-5-ethyl-4-formyl-2-isopropyl-1H-pyrrole-3-carboxylic Acid Methyl Ester (Compound 45): 1-Benzyl-5-ethyl-2-isopropyl-1H-pyrrole-3-carboxylic acid methyl ester (Compound 44, 1.4 g, 5.6 mmol) in 5 ml of DMF was added to the solution of POCl₃ (1.72 g, 11.2 mmol) in 5 ml of DMF at 0° C. The reaction solution was stirred at 90° C. for 18 hours and cooled to room temperature. The reaction was quenched with H₂O, extracted with ethyl acetate, washed with brine, dried over Na₂SO₄, and concentrated under reduced pressure. The title product was purified by column chromatography on silica gel with 10% EtOAc/Hex as solid.

1H-NMR (CDCl₃): 1.07 (t, J=7.33 Hz, 3H), 1.22 (d, J=7.03 Hz, 6H), 2.87 (q, J=7.33 Hz, 2H), 3.45 (hept, J=7.03 Hz, 1H), 3.86 (s, 3H), 5.17 (s, 2H), 6.88 (d, J=7.13 Hz, 2H), 7.21-7.35 (m, 3H), 10.24 (s, 1H).

1-Benzyl-5-ethyl-2-isopropyl-4-vinyl-1H-pyrrole-3-carboxylic Acid Methyl Ester (Compound 46). General Procedure 5: n-BuLi (2.5M in hex, 0.88 ml, 2.2 mmol) was added dropwise to the suspension of methyl triphenylphosphonium bromide (734 mg, 2.06 mmol) in 10 ml of THF at 0° C. and stirred for 20 mins at 0° C. A solution of 1-benzyl-5-ethyl-4-formyl-2-isopropyl-1H-pyrrole-3-carboxylic acid methyl ester (compound 45, 450 mg, 1.37 mmol) in 10 ml of THF was transferred into the above reaction. The resulting solution was stirred at ROOM TEMPERATURE for 2 hours and quenched with H₂O, extracted with DCM, washed with brine, dried over Na₂SO₄, and concentrated under reduced pressure. The title product was purified by column chromatography on silica gel with 4 to10% EtOAc/Hex as solid.

1H-NMR (CDCl₃): 1.06 (t, J=7.33 Hz, 3H), 1.22 (d, J=7.03 Hz, 6H), 2.59 (q, J=7.33Hz, 2H), 3.26 (hept, J=7.03 Hz, 1H), 3.82 (s, 3H), 5.15 (s, 2H), 5.18-5.22 (m, 2H), 6.82-6.95 (m.3H), 7.21-7.35 (m, 3H).

Compound 47 was prepared by General Procedure 5.

1-Benzyl-4-(but-1-enyl)-5-ethyl-2-isopropyl-1H-pyrrole-3-carboxylic acid methyl ester (Compound 47) was prepared as a mixture of E and Z isomers using n-BuLi (2.5 M in hex, 1.25 ml, 3.12 mmol), propyl triphenylphosphonium bromide (1.10 g, 2.86 mmol) and 1-benzyl-5-ethyl-4-formyl-2-isopropyl-1H-pyrrole-3-carboxylic acid methyl ester (Compound 45, 520 mg, 1.56 mmol) after purification by silica gel chromatography.

1H-NMR (CDCl₃): 0.9-1.0 (m, 6H), 1.15-1.27 (m, 6H), 1.99 (m, 1.5H), 2.20 (m, 0.5H), 2.39 (q, J=7.62Hz, 1.5H), 2.56 (q, J=7.62Hz, 0.5H), 3.12-3.20 (m, 1H), 3.76(s, 2.25H), 3.81 (s, 0.75H), 5.15 (s, 2H), 5.10-5.22 (m, 0.75H), 5.24-5.35 (m, 0.25H), 6.25-6.34 (m, 0.75H), 6.48-6.58 (m, 0.25H), 6.87 (d, J=6.74 Hz, 2H), 7.21-7.35 (m, 3H).

1-Benzyl-4,5-ethyl-2-isopropy-1H-pyrrole-3-carboxylic acid methyl ester (Compund 48). General Procedure 6: 1-Benzyl-5-ethyl-2-isopropyl-4-vinyl-1H-pyrrole-3-carboxylic acid methyl ester (Compound 46, 240 mg, 0.74 mmol) was dissolved in 20 ml of THF with 0.1 ml of TEA and 35 mg of 10%Pd/C was added. The reaction mixture was stirred under H₂ balloon for one hour. After the solid was filtered thought a pad of celite, the filtrate was concentrated to afford the title compound.

1H-NMR (CDCl₃): 0.92 (t, J=7.62 Hz, 3H), 1.06 (t, J=7.33 Hz, 3H), 1.22 (d, J=7.03 Hz, 6H), 2.35 (q, J=7.62 Hz, 2H), 2.59 (q, J=7.33 Hz, 2H), 3.31 (hept, J=7.03 Hz, 1H), 3.73 (s, 3H), 5.04 (s, 2H), 6.78 (d, J=6.74 Hz, 0.2H), 7.10-7.25 (m, 3H).

Compound 48 was also prepared by General Procedure 6

1-Benzyl-4-butyl-5-ethyl-2-isopropyl-1H-pyrrole-3-carboxylic acid methyl ester (Compound 48) was prepared with a mixture of (E)- and (Z)-1-benzyl-4-but-1-enyl)-5-ethyl-2-isopropyl-1H-pyrrole-3-carboxylic acid methyl ester (Compound 47, 210 mg, mmol) and 10% Pd/C (55 mg) in THF (20 ml) and TEA (0.1 ml) under H₂ balloon.

1H-NMR (CDCl₃): 0.93 (t, J=7.33 Hz, 3H), 0.99 (t, J=7.62 Hz, 3H), 1.22 (d, J=7.03 Hz, 6H), 1.25-1.45 (m, 4H), 2.63 (q, J=7.62 Hz, 2H), 2.55-2.63 (m, 2H), 3.41 (hept, J=7.03 Hz, 1H), 3.80 (s, 3H), 5.12 (s, 2 H), 6.84 (d, J=6.74 Hz, 0.2H), 7.20-7.35 (m, 3H).

1-Benzyl-4,5-ethyl-2-isopropy-1H-pyrrole-3-carboxylic Acid (Compound 50). General Procedure 7: 1-Benzyl-4,5-ethyl-2-isopropy-1H-pyrrole-3-carboxylic acid methyl ester (Compound 48, 210 mg, 0.7 mmol) was treated with 5N aqueous NaOH (4 ml) in MeOH (10 ml) at 90° C. for 7 days. The reaction solution was cooed to room temperature, neutralized with 10% aqueous HCl, extracted with ether, dried over Na₂SO₄, and concentrated under reduced pressure to yield the title compound with unreacted starting material.

1H-NMR (CDCl₃): 0.92 (t, J=7.62 Hz, 3H), 1.07 (t, J=7.33 Hz, 3H), 1.23 (d, J=7.03 Hz, 6H), 2.36 (q, J=7.62 Hz, 2H), 2.59 (q, J=7.33 Hz, 2H), 3.33 (hept, J=7.03 Hz, 1H), 5.06 (s, 2H), 6.78 (d, J=6.74 Hz,.2H), 7.12-7.25 (m, 3H).

1-Benzyl-4-butyl-5-ethyl-2-isopropyl-1H-pyrrole-3-carboxylic acid (Compound 51) was prepared with 1-benzyl-4-butyl-5-ethyl-2-isopropy-1H-pyrrole-3-carboxylic acid methyl ester (Compound 49, 200 mg, 0.58 mmol) and 5N NaOH (4 ml) in MeOH (10 ml) at 90° C. for 7 days according to general procedure 7 as a mixture with unreacted starting material.

1H-NMR (CDCl₃): 0.95-0.99 (m, 6H), 1.22 (d, J=7.03 Hz, 6H), 1.25-1.45 (m, 4H), 2.35 (q, J=7.62 Hz, 2H), 2.50-2.62 (m, 2H), 3.30 (m, 1H), 5.05 (s, 2 H), 6.87 (d, J=6.74 Hz, 0.2H), 7.20-7.35 (m, 3H).

Compounds 52 and 53 were prepared by General Procedure 4

1-Benzyl-4,5-ethyl-2-isopropy-1H-pyrrole-3-carboxylic acid 3,4-difluoro-benzylamide (compound 52) was prepared as a white solid from 1-benzyl-4,5-ethyl-2-isopropy-1H-pyrrole-3-carboxylic acid (179 mg, 0.6 mmol), EDCI (170 mg, 0.89 mmol), DMAP (122 mg, 1 mmol), and 3,4-difluorobenzylamine (103 mg, 0.70 mmol).

1H-NMR (CDCl₃): 0.97 (t, J=7.62 Hz, 3H),): 1.09 (t, J=7.62 Hz, 3H), 1.21 (d, J=7.33 Hz, 6H), 2.41 (q, J=7.62 Hz, 2H), 2.49 (q, J=7.62 Hz, 2H), 3.03 (hept, J=7.33 Hz, 1H), 4.56 (d, J=6.15 Hz, 2H), 5.06 (s, 2H), 5.95 (bs, 1H), 6.85 (d, J=6.84 Hz, 2H), 7.07-7.36 (m, 6H).

1-Benzyl-4-butyl-5-ethyl-2-isopropy-1H-pyrrole-3-carboxylic acid 3,4-difluoro-benzylamide (compound 53) was prepared as a white solid from 1-benzyl-4-butyl-5-ethyl-2-isopropy-1H-pyrrole-3-carboxylic acid (Compound 51, 164 mg, 0.5 mmol), EDCI (170 mg, 0.89 mmol), DMAP (122 mg, 1 mmol), and 3,4-difluorobenzylamine (103 mg, 0.70 mmol).

1H-NMR (CDCl₃): 0.87 (t, J=7.32 Hz, 3H),): 0.96 (t, J=7.32 Hz, 3H), 1.21 (d, J=7.03 Hz, 6H), 1.22-1.44 (m, 4H), 2.35-2.45 (m, 4H), 3.03 (hept, J=7.03 Hz, 1H), 4.56 (d, J=5.86 Hz, 2H), 5.06 (s, 2H), 5.95 (bs, 1H), 6.83 (d, J=6.84 Hz, 2H), 7.05-7.36 (m, 6H).

| Compound | |
|---|---|
| Number | Structure |
| 33 | 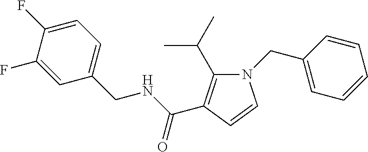 |
| 34 | 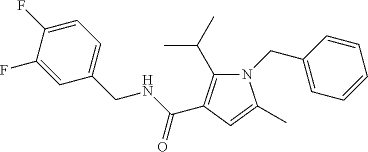 |
| 35 | 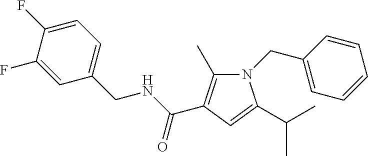 |
| 36 | 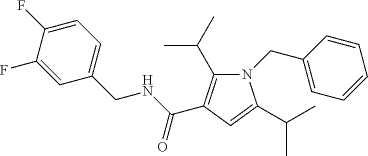 |
| 37 | 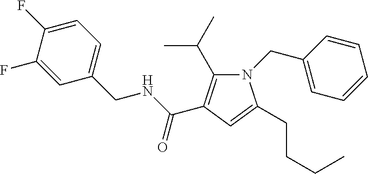 |
| 38 | 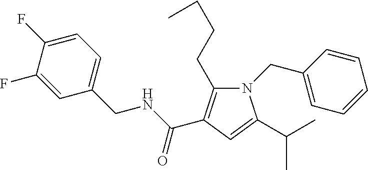 |
| 39 | 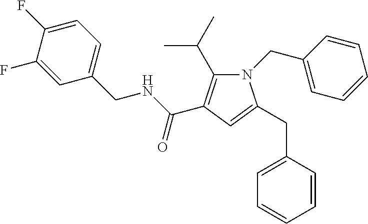 |
| 40 | 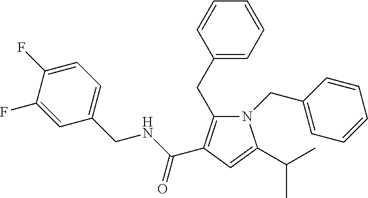 |
| 41 | 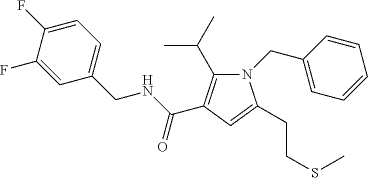 |
| 42 | 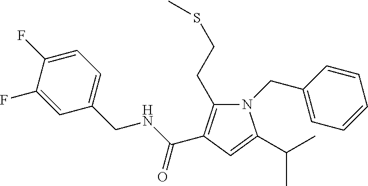 |
| 52 | 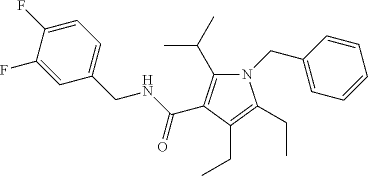 |
| 53 | 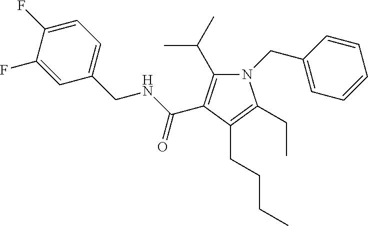 |

What is claimed is:

1. A compound represented by: 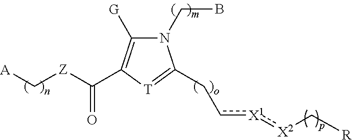
wherein a dashed line represents the presence or absence of a bond;
A and B are independently stable substituted or unsubstituted aryl, or substituted or unsubstituted heteroaryl, wherein A and B independently have a formula C₁₋₁₂H₀₋₂₉N₀₋₄O₀₋₄S₀₋₄F₀₋₆Cl₀₋₂Br₀₋₂I₀₋₂;
m, n, o, and p are independently 0, 1, 2, or 3;
R is H; C₁₋₈ non-linear alkyl; C₁₋₈ acyl; C₁₋₈ alkoxycarbonyl; or a stable substituted or unsubstituted heterocycle or phenyl having a formula C₁₋₁₂H₀₋₂₉N₀₋₄O₀₋₃S₀₋₃F₀₋₆Cl₀₋₂Br₀₋₂I₀₋₂;
Z is CH₂, O, N, or S;
T is CH or N or an alkyl having from 1 to 4 carbon atoms;
G is H, phenyl or is a moiety having from 1 to 6 carbon atoms selected from: alkyl wherein one of the carbons may be substituted with S, fluoroalkyl, acyl, hydroxyalkyl, amino or substituted or unsubstituted heteroaryl; and
X¹ and X² are independently a bond, 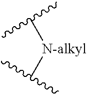
having from 1 to 4 carbon atoms, 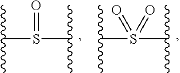
C═O, —CH═, ═CH—, NH, ═N—, —N═, S, or O;
provided that both X¹ and X² are not bonds.

2. The compound of claim 1 represented by: 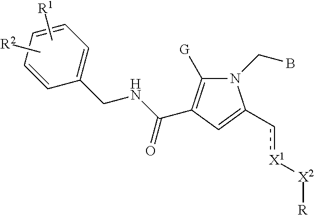
wherein R¹ and R² are independently H, F, Cl, NO₂, methyl, ethyl, n-propyl, or iso-propyl;
B is phenyl or pyridinyl which is unsubstituted, or has 1 or 2 substituents independently selected from F, Cl, NO₂, methyl, ethyl, n-propyl, and iso-propyl;
X¹ and X² are independently a bond, ═N, O, or ═CH—;
R is C₁₋₅ alkyl; or R is a phenyl or a heterocyclic group which is unsubstituted or has 1 or 2 substituents independently selected from: F, Cl, NO₂, methyl, ethyl, n-propyl, and iso-propyl.

3. The compound of claim 2 wherein X¹-X² are selected from ═C—, ═N—O—, and O.

4. The compound of claim 3 wherein B is unsubstituted phenyl.

5. The compound of claim 3 wherein B is unsubstituted pyridinyl.

6. The compound of claim 5 wherein R is iso-propyl.

7. The compound of claim 4 wherein R is methylphenyl.

8. The compound of claim 4 wherein R is n-butyl.

9. The compound of claim 3 wherein R¹ and R² are independently H, methyl, F, or NO₂.

10. The compound of claim 1 wherein B is phenyl.

11. The compound of claim 1 wherein B is pyridinyl.

12. The compound of claim 1 wherein A is substituted phenyl.

13. The compound of claim 1 wherein Z is N or CH₂.

14. The compound of claim 1 wherein T is CH.

15. The compound of claim 1 wherein m is 0.

16. The compound of claim 1 wherein n is 1.

17. Use of a compound according to any one of claims 1-16 in the manufacture of a medicament for the treatment of a disease or condition in a mammal, said disease or condition selected from glaucoma, dry eye, angiogenesis, cardiovascular conditions and diseases, wounds, and pain.

18. The method of claim 17 wherein the mammal is a human.

19. A method of treating a disease or condition comprising administering a compound according to any one of claims 1-16 to a mammal in need thereof, said disease or condition selected from glaucoma, dry eye, angiogenesis, cardiovascular conditions and diseases, wounds, and pain.

20. The method of claim 19 wherein the mammal is a human.
